(19) United States
(12) Patent Application Publication
(10) Pub. No.: US 2008/0233583 A1
(43) Pub. Date: Sep. 25, 2008

(54) BIOMARKERS FOR PREECLAMPSIA

(75) Inventors: Susan J. Fisher, San Francisco, CA (US); Virginia D. Winn, Denver, CO (US); Ronit Haimov-Kochman, Mevasseret Zion (IL)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: REGENTS OF THE UNIVERSITY OF CALIFORNIA, Oakland, CA (US)

(21) Appl. No.: 12/034,458

(22) Filed: Feb. 20, 2008

Related U.S. Application Data
(60) Provisional application No. 60/890,829, filed on Feb. 20, 2007.

(51) Int. Cl. C12Q 1/68; G01N 33/566; G01N 33/50; G01N 33/567

(52) U.S. Cl. 435/6; 436/501; 436/94; 435/7.21

(57) ABSTRACT

The present invention provides methods for predicting the development of and diagnosing preeclampsia, providing a prognosis, and predicting recurrence of the disease using molecular markers that are overexpressed or underexpressed in preeclampia. Also provided are methods to identify compounds that are useful for the treatment or prevention of preeclampsia.

### CROSS-REFERENCES TO RELATED APPLICATIONS

The present application claims priority to U.S. Ser. No. 60/890,829, filed Feb. 20, 2007, herein incorporated by reference in its entirety.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### BACKGROUND OF THE INVENTION

Preeclampsia is a pregnancy-specific, multisystem disorder that is characterized by the develepoment of hypertension and proteinuria. The incidence of this disorder is approximately 5 to 7 percent of pregnancies, resulting in about 24 cases per 1000 deliveries in the United States. Complications arising from the hypertension attendant to preeclampsia are one of the leading causes of pregnancy-related deaths. Among the risks associated with preeclampsia are placental abruption, acute renal failure, cerebrovascular and cardiovascular complications, disseminated intravascular coagulation, and maternal death. See, generally, Wagner, L. K., “Diagnosis and Management of Preeclampsia”, American Family Physician, 70: 2317-2324, 2004.

Among the criteria for diagnosis of preeclampsia is the onset of elevated blood pressure and proteinuria after 20 weeks of gestation. Specifically, these criteria include a blood pressure 140 mm Hg or higher systolic or 90 mm Hg diastolic after 20 weeks of gestation in a woman with previously normal blood pressure. Increased proteinuria corresponds to 0.3 grams or more of protein in a 24 hour urine collection; this generally corresponds with 1+ or greater on a urine dipstick test. More severe preeclampsia presents with more substantial blood pressure elevations and higher degrees of proteinuria. Thus, severe preeclampsia may be indicated by 160 mm Hg or higher systolic or 110 mm Hg or higher diasystolic on two occasions at least six hours apart in a woman on bed rest. In severe cases, proteinuria may be elevated to 5 grams or more of protein in a 24 hour urine collection or 3+ or greater on urine dipstick testing of two random samples collected at least four hours apart. Other features of severe preeclampsia include: oliguria (less than 500 mL of urine in 24 hours), cerebral or visual disturbances, pulmonary edema or cycnosis, epigastric or right upper quadrant pain, impaired liver function, thrombocytopenia, and intrauterine growth restriction. See, generally, Wagner, L. K., “Diagnosis and Management of Preeclampsia”, American Family Physician, 70: 2317-2324, 2004.

Although diagnostic criteria for preeclampsia exist, the diagnosis of preeclampsia may be complicated by other conditions associated with pregnancy. For instance, a diagnosis of preeclampsia may be confounded by other hypertensive disorders of pregnancy. Such hypertensive disorders include chronic hypertension, preeclampsia-eclampsia, preeclampsia superimposed on chronic hypertension, and gestational hypertension. Thus, a physician must determine how a patient's particular set of symptoms fits into the overall spectrum of hypertensive disorders of pregnancy in order to devise an effective course of treatment. In addition, there is currently no way to predict which 5-7 percent of women will develop preeclampsia, before the onset of symptoms. Reliable prediction would allow physicians to taylor an individual woman's care in order to prevent the eventual onset of preeclampsia or to reduce the consequences of the disease.

Given the severe and even life threatening consequences of preeclampsia, prediction of a woman's risk of developing the disease, as well as, early and unambiguous diagnosis and effective treatment strategies are imperative. This invention satisfies these and other needs.

### BRIEF SUMMARY OF THE INVENTION

Human placentation entails the remarkable integration of fetal and maternal cells into a single functional unit. In the basal plate region (the maternal-fetal interface) of the placenta, fetal cytotrophoblasts from the placenta invade the uterus and remodel the resident vasculature while avoiding maternal immune rejection. Knowing the molecular bases for these unique cell-cell interactions is important for understanding how this specialized region functions during normal and abnormal pregnancies. Because the maternal-fetal interface is a site of known anatomical defect in preeclampsia, we undertook a global analysis of the gene expression profiles at the maternal-fetal interface from preeclampsia patients and a control group. Basal plate biopsy specimens were obtained from placentas at the conclusion of pregnancies. RNA was isolated, processed and hybridized to HG-U133A&B Affymetrix GeneChips. From these studies, genes which were up- or down regulated in preeclampsia were identified. Subsequent analyses using Q-PCR and immunolocalization approaches validated a portion of these results. Many of the differentially expressed genes are known in other contexts to be involved in differentiation, motility, transcription, immunity, angiogenesis, extracellular matrix dissolution or lipid metabolism. These data provide a reference set for use as biomarkers of preeclampsia (individually or in combination) and can serve as targets for the prediction, diagnosis, prevention, and treatment of preeclampsia.

### BRIEF DESCRIPTION OF THE DRAWINGS

FIG. 1 shows a schematic diagram of the maternal-fetal interface or basal plate.

FIG. 2 shows a model for the development of preeclampsia.

FIG. 3 shows a heat map, gene descriptions, and induction levels of genes upregulated in preeclampsia.

FIG. 4 shows a heat map, gene descriptions, and induction levels of genes down regulated in preeclampsia.

FIG. 5 shows the induction levels of mRNA for nine genes identified as either upregulated or downregulated during preeclampsia, as a function of gestational age in normal subjects and preeclampsia patients.

FIG. 6 shows increased levels of SigLec-6 protein in preeclampsia placentas as compared to normal preterm placentas by immunofluroescence.

FIG. 7 shows a model preeclampsia clinical flow sheet that illustrates one of the clinical applications of the invention.

FIG. 8: PAPPA2 is increased in the basal plate from PE placentas compared to controls. Gestational ages are represented as (week.day) and in general were loaded from left to right by increasing GA. Serum is the positive control for PAPPA2 from pregnant term woman. The Arrow heads are the 2 placentas (#129 and 130) that had molecular signatures on the arrays that were not consistent with the rest of the samples. The graph is the normalized densitometry results of the immunoblot above. The mean and SEM for the PE samples is 21.16±3.1 and for the PTL samples 11.13+1.1 which gives roughly a 2-fold change. The fold change at the RNA level based on the microarray data was 2.5 fold and for the Q-PCR data was also ˜2-fold. There is clearly a significant difference in the levels of PAPPA2 in PE versus PTL samples and this difference is most dramatic at the earlier gestational ages (<33 weeks). To determine which cell types in the basal plate region express PAPPA2 and account for the fold change seen both at the RNA and protein level we performed localization studies via IHC.

FIG. 9: The next Figures are representative IHC for PAPPA2 for placental basal plate biopsies. At this point we have stained PE n=6 and PTL n=4. 10% formalin fixed and paraffin-embedded tissues were serially sectioned. Antigen retrieval was with citric acid buffer in a steam cooker for 20 min. Primary antibody was used at 1:30,000. The insert is the negative control of no primary antibody. CK7 labels all trophoblast cells. HLA-G labels the invasive cytotrophoblast cells within the basal plate. The PTL sample shows PAPPA2 staining in the invasive CTB population and not the syncytial CTBs lining the chorionic villi (CV). AV refers to the anchoring villi. In contrast, the PE 24 wk sample does have staining of the invasive AND syncytial CTBs lining the chorionic villi.

FIG. 10: We see a similar pattern at 32-33 weeks with increased syncytial CTB staining in PE compared to PTL. You can also see clearly that the syncytial lining of the basal plate is staining for PAPPA2 (arrows). Given PAPPA2 is expressed by the sCTB one would anticipate that this difference would be reflected in the serum.

### DETAILED DESCRIPTION OF THE INVENTION

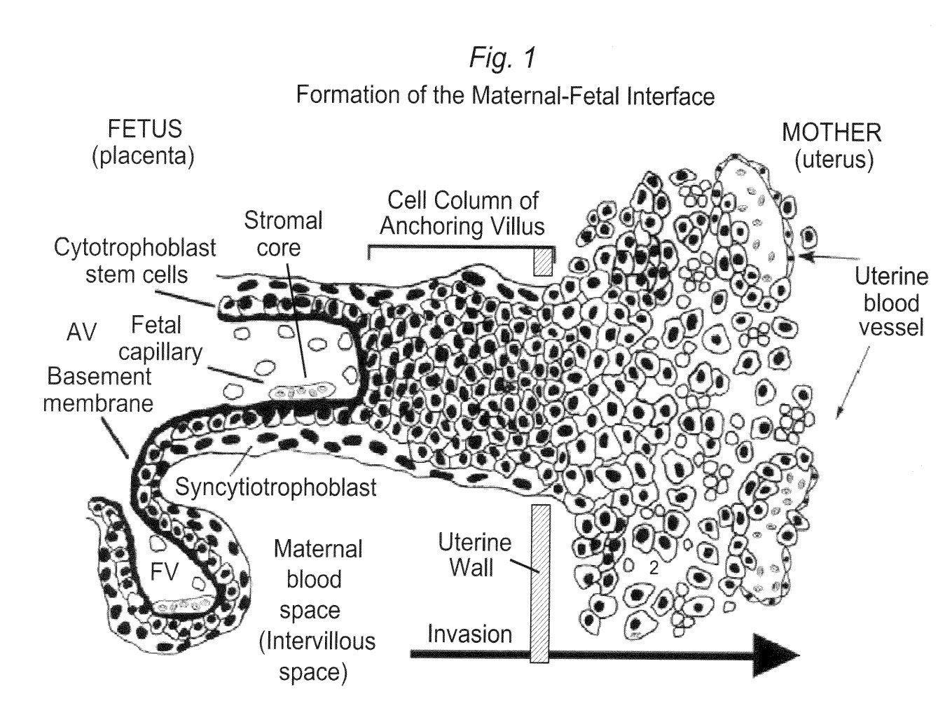

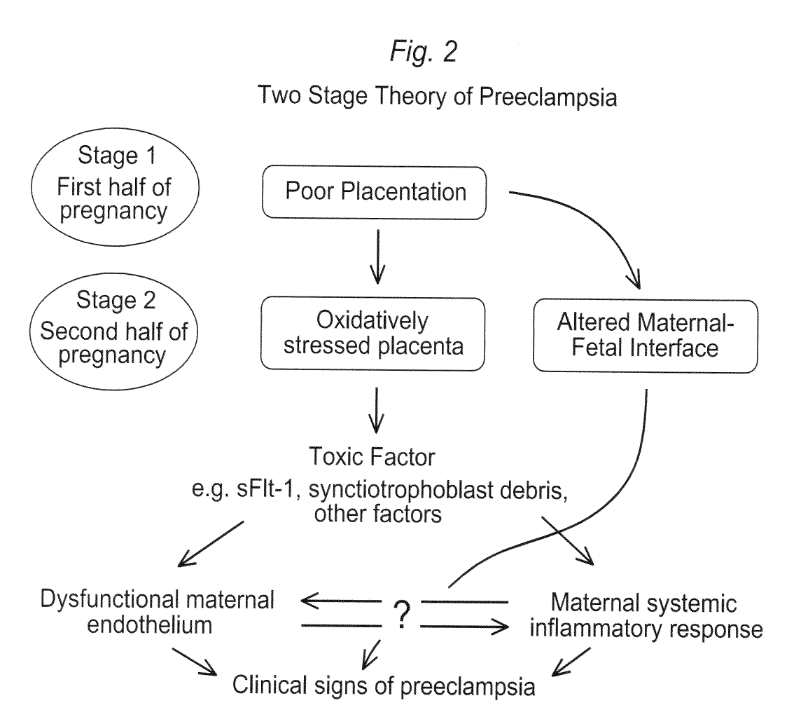

Survival and growth of the fetus require normal development of the placenta, which in humans involves the formation of a transient organ with both maternal and fetal contributions. Specifically, invasive cytotrophoblasts (CTBs), components of anchoring chorionic villi, attach to and invade the maternal decidua. A subset of these cells remodel the uterine vasculature, which they also occupy (FIG. 1). This process primarily occurs during the second trimester of pregnancy. The region where maternal and fetal cells coexist is termed the basal plate or maternal-fetal interface, and its proper formation and function are required for normal pregnancy outcome. Previously, we have performed gene expression studies to determine the profile of genes expressed during gestational stages and at term at the human maternal-fetal interface in normal pregnancies (see Winn, V. D. et al., Endocrinology, 148: 1059-1079, 2007).

As discussed above, preeclampsia is a serious and potentially life threatening complication of many pregnancies defined by hypertension and proteinuria. Preeclampsia has been characterized by many investigators as a syndrome of multiple organ failure involving the liver, kidney, and lung, as well as, coagulatory and neural systems. The consensus is emerging that preeclampsia is a complex polygenetic trait in which maternal and fetal genes, as well as environmental factors, are involved (see FIG. 1). While the precise mechanism of this disorder is unknown, the maternal-fetal interface or basal plate is a site of known anatomical defect in preeclampsia. Thus, we have performed a global analysis of gene expression profiles at the human maternal-fetal interface in both normal patients and patients with preeclampsia in order to better understand how the molecular components of the dialogue that takes place between maternal and fetal cells in the human basal plate region may be altered during preeclampsia. In the process of studying these changes in gene expression, we have been able to identify useful biomarkers for preeclampsia as well as targets for therapeutic intervention of this disorder.

### Definitions

Preclampsia refers generally to a pregnancy-specific, multisystem disorder that is characterized by the development of hypertension and proteinuria. Among the signs and symptoms of preeclampsia are an elevated blood pressure of >140/90 (mild) or >160/110 (severe) and proteinuria of >300 mg/24 hours (mild) or 5 gm/24 hours (severe). Other symptoms of preclampsia include edema, RUQ/epigastric pain, headache, visual changes, hemolysis, elevated liver tests, low platelets, oligouria, pulmonary edema, and seizure. See, generally, Williams Obstetrics, 22nd edition, 2005.

The “basal plate” region or the “maternal-fetal interface” refers generally to the region of the placenta where fetal cytotrophoblasts from the placenta invade the uterus resulting in remodeling of the resident vasculature. See, generally, Moore's The Developing Human: Clinically Oriented Embryology, 6th edition, 1998.

The term “marker” or “biomarker” refers to a molecule (typically protein, nucleic acid, carbohydrate, or lipid) that is expressed in a cell, expressed on the surface of a cell or secreted by a cell and which is useful for the prediction of the risk of developing preeclampsia, for diagnosis of preeclampsia, for providing a prognosis of preeclampsia, and for preferential targeting of a pharmacological agent in the treatment of preeclampsia. Such biomarkers are molecules that are overexpressed in preeclampsia in comparison to a normal pregnancy, for instance, 1-fold overexpression, 2-fold overexpression, 3-fold overexpression, or more. Alternatively, such biomarkers are molecules that are underexpressed in preeclampsia in comparison to a normal pregnancy, for instance, 1-fold underexpression, 2-fold underexpression, 3-fold underexpression, or more. Further, a marker can be a molecule that is inappropriately synthesized in preeclampsia, for instance, a molecule that contains deletions, additions or mutations in comparison to the molecule expressed on a normal cell.

It will be understood by the skilled artisan that markers may be used singly or in combination with other markers for any of the uses, e.g., prediction, diagnosis, or prognosis of preeclampsia, disclosed herein.

“Biological sample” includes sections of tissues such as biopsy and autopsy samples, and frozen sections taken for histologic purposes. Such samples include blood and blood fractions or products (e.g., serum, plasma, platelets, red blood cells, and the like), sputum, cervicovaginal fluid, lymph and tongue tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, Mouse; rabbit; or a bird; reptile; or fish.

A “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (e.g., placenta, skin, colon, prostate, kidney, bladder, lymph node, liver, bone marrow, blood cell, etc.), the size and type of the tumor (e.g., solid or suspended, blood or ascites), among other factors. Representative biopsy techniques include, but are not limited to, excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy can require a “core-needle biopsy”, or a “fine-needle aspiration biopsy” which generally obtains a suspension of cells from within a target tissue. In the case of placental tissue, biopsies are generally conducted post-delivery. Biopsy techniques are discussed, for example, in Harrison 's Principles of Internal Medicine, Kasper, et al., eds., 16th ed., 2005, Chapter 70, and throughout Part V.

The terms “overexpress,” “overexpression” or “overexpressed” or “upregulated” interchangeably refer to a protein or nucleic acid (RNA) that is transcribed or translated at a detectably greater level, usually in a preeclampsia patient, in comparison to a patient with a normal pregnancy. The term includes overexpression due to transcription, post transcriptional processing, translation, post-translational processing, cellular localization (e.g., organelle, cytoplasm, nucleus, cell surface), and RNA and protein stability, as compared to a control. Overexpression can be detected using conventional techniques for detecting mRNA (i.e., RT-PCR, PCR, hybridization) or proteins (i.e., ELISA, immunohistochemical techniques). Overexpression can be 10%, 20%, 30%, 40%, 50%, 60%, 70%, 80%, 90% or more in comparison to a normal cell. In certain instances, overexpression is 1-fold, 2-fold, 3-fold, 4-fold or more higher levels of transcription or translation in comparison to a control.

The terms “underexpress,” “underexpression” or “underexpressed” or “downregulated” interchangeably refer to a protein or nucleic acid that is transcribed or translated at a detectably lower level, usually in a preeclampsia patient, in comparison to a patient with a normal pregnancy. The term includes underexpression due to transcription, post transcriptional processing, translation, post-translational processing, cellular localization (e.g., organelle, cytoplasm, nucleus, cell surface), and RNA and protein stability, as compared to a control. Underexpression can be detected using conventional techniques for detecting mRNA (i.e., RT-PCR, PCR, hybridization) or proteins (i.e., ELISA, immunohistochemical techniques). Underexpression can be 10%, 20%, 30%, 40%, 50%, 60%, 70%, 80%, 90% or less in comparison to a control. In certain instances, underexpression is 1-fold, 2-fold, 3-fold, 4-fold or more lower levels of transcription or translation in comparison to a control.

The term “differentially expressed” or “differentially regulated” refers generally to a protein or nucleic acid that is overexpressed (upregulated) or underexpressed (downregulated) in one sample compared to at least one other sample, generally in a preeclampsia patient, in comparison to a patient with a normal pregnancy, in the context of the present invention.

“Therapeutic treatment” refers to drug therapy, hormonal therapy, immunotherapy, and biologic (targeted) therapy.

By “therapeutically effective amount or dose” or “sufficient amount or dose” herein is meant a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1987-2005, Wiley Interscience)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

“RNAi molecule” or an “siRNA” refers to a nucleic acid that forms a double stranded RNA, which double stranded RNA has the ability to reduce or inhibit expression of a gene or target gene when the siRNA expressed in the same cell as the gene or target gene. “siRNA” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 nucleotides in length, e.g., 20, 21, 22, 23, 24, 25, 26, 27, 28, 29, or 30 nucleotides in length.

An “antisense” polynucleotide is a polynucleotide that is substantially complementary to a target polynucleotide and has the ability to specifically hybridize to the target polynucleotide.

Ribozymes are enzymatic RNA molecules capable of catalyzing specific cleavage of RNA. The composition of ribozyme molecules preferably includes one or more sequences complementary to a target mRNA, and the well known catalytic sequence responsible for mRNA cleavage or a functionally equivalent sequence (see, e.g., U.S. Pat. No. 5,093,246, which is incorporated herein by reference in its entirety). Ribozyme molecules designed to catalytically cleave target mRNA transcripts can also be used to prevent translation of subject target mRNAs.

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants” and nucleic acid sequences encoding truncated forms of a protein. Similarly, a particular protein encoded by a nucleic acid implicitly encompasses any protein encoded by a splice variant or truncated form of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. Nucleic acids can be truncated at the 5′ end or at the 3′ end. Polypeptides can be truncated at the N-terminal end or the C-terminal end. Truncated versions of nucleic acid or polypeptide sequences can be naturally occurring or recombinantly created.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M). See, e.g., Creighton, Proteins (1984).

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., supra.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.- 2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. Antibodies can be polyclonal or monoclonal, derived from serum, a hybridoma or recombinantly cloned, and can also be chimeric, primatized, or humanized.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

In one embodiment, the antibody is conjugated to an “effector” moiety. The effector moiety can be any number of molecules, including labeling moieties such as radioactive labels or fluorescent labels, or can be a therapeutic moiety. In one aspect the antibody modulates the activity of the protein.

The nucleic acids of the differentially expressed genes of this invention or their encoded polypeptides refer to all forms of nucleic acids (e.g., gene, pre-mRNA, mRNA) or proteins, their polymorphic variants, alleles, mutants, and interspecies homologs that (as applicable to nucleic acid or protein): (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200, 500, 1000, or more amino acids, to a polypeptide encoded by a referenced nucleic acid or an amino acid sequence described herein; (2) specifically bind to antibodies, e.g., polyclonal antibodies, raised against an immunogen comprising a referenced amino acid sequence, immunogenic fragments thereof, and conservatively modified variants thereof; (3) specifically hybridize under stringent hybridization conditions to a nucleic acid encoding a referenced amino acid sequence, and conservatively modified variants thereof, (4) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, to a reference nucleic acid sequence. A polynucleotide or polypeptide sequence is typically from a mammal including, but not limited to, primate, e.g., human; rodent, e.g., rat, mouse, hamster; cow, pig, horse, sheep, or any mammal. The nucleic acids and proteins of the invention include both naturally occurring or recombinant molecules. Truncated and alternatively spliced forms of these antigens are included in the definition.

The phrase “specifically (or selectively) binds” when referring to a protein, nucleic acid, antibody, or small molecule compound refers to a binding reaction that is determinative of the presence of the protein or nucleic acid, such as the differentially expressed genes of the present invention, often in a heterogeneous population of proteins or nucleic acids and other biologics. In the case of antibodies, under designated immunoassay conditions, a specified antibody may bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

The phrase “functional effects” in the context of assays for testing compounds that modulate a marker protein includes the determination of a parameter that is indirectly or directly under the influence of a biomarker of the invention, e.g., a chemical or phenotypic effect such as altered transcriptional activity of SigLec-6 in the leptin signaling pathway and the downstream effects of such proteins on cellular metabolism and growth. A functional effect therefore includes ligand binding activity, transcriptional activation or repression, the ability of cells to proliferate, the ability to migrate, among others. “Functional effects” include in vitro, in vivo, and ex vivo activities.

By “determining the functional effect” is meant assaying for a compound that increases or decreases a parameter that is indirectly or directly under the influence of a biomarker of the invention, e.g., measuring physical and chemical or phenotypic effects. Such functional effects can be measured by any means known to those skilled in the art, e.g., changes in spectroscopic characteristics (e.g., fluorescence, absorbance, refractive index); hydrodynamic (e.g., shape), chromatographic; or solubility properties for the protein; ligand binding assays, e.g., binding to antibodies; measuring inducible markers or transcriptional activation of the marker; measuring changes in enzymatic activity; the ability to increase or decrease cellular proliferation, apoptosis, cell cycle arrest, measuring changes in cell surface markers. The functional effects can be evaluated by many means known to those skilled in the art, e.g., microscopy for quantitative or qualitative measures of alterations in morphological features, measurement of changes in RNA or protein levels for other genes expressed in placental tissue, measurement of RNA stability, identification of downstream or reporter gene expression (CAT, luciferase, β-gal, GFP and the like), e.g., via chemiluminescence, fluorescence, colorimetric reactions, antibody binding, inducible markers, etc.

“Inhibitors,” “activators,” and “modulators” of the markers are used to refer to activating, inhibitory, or modulating molecules identified using in vitro and in vivo assays of preeclampsia biomarkers. Inhibitors are compounds that, e.g., bind to, partially or totally block activity, decrease, prevent, delay activation, inactivate, desensitize, or down regulate the activity or expression of preeclampsia biomarkers. “Activators” are compounds that increase, open, activate, facilitate, enhance activation, sensitize, agonize, or up regulate activity of preeclampsia biomarkers, e.g., agonists. Inhibitors, activators, or modulators also include genetically modified versions of preeclampsia biomarkers, e.g., versions with altered activity, as well as naturally occurring and synthetic ligands, antagonists, agonists, antibodies, peptides, cyclic peptides, nucleic acids, antisense molecules, ribozymes, RNAi molecules, small organic molecules and the like. Such assays for inhibitors and activators include, e.g., expressing preeclampsia biomarkers in vitro, in cells, or cell extracts, applying putative modulator compounds, and then determining the functional effects on activity, as described above.

Samples or assays comprising preeclampsia biomarkers that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition of preeclampsia biomarkers is achieved when the activity value relative to the control is about 80%, preferably 50%, more preferably 25-0%. Activation of preeclampsia biomarkers is achieved when the activity value relative to the control (untreated with activators) is 110%, more preferably 150%, more preferably 200-500% (i.e., two to five fold higher relative to the control), more preferably 1000-3000% higher.

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, peptide, circular peptide, lipid, fatty acid, siRNA, polynucleotide, oligonucleotide, etc., to be tested for the capacity to directly or indirectly modulate preeclampsia biomarkers. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

### Predictive, Diagnostic, and Prognostic Methods

The present invention provides methods of predicting, diagnosing or providing prognosis of preeclampsia by detecting the expression of markers overexpressed or underexpressed in preeclampsia. Prediction and diagnosis involve determining the level of one or more preeclampsia biomarker polynucleotide or the corresponding polypeptides in a patient or patient sample and then comparing the level to a baseline or range. Typically, the baseline value is representative of levels of the polynucleotide or nucleic acid in a healthy person not suffering from, or destined to develop, preeclampsia, as measured using a biological sample such as a placental biopsy or a sample of a bodily fluid (e.g., blood, salvia, cervicovaginal fluid, or urine). Variation of levels of a polynucleotide or corresponding polypeptides of the invention from the baseline range (either up or down) indicates that the patient has an increased risk of developing preeclampsia or an increased risk of its recurrence.

As used herein, the term “prediction” refers to providing a measure of relative risk for developing preeclampsia later in pregnancy in a patient who is asymptomatic. As used herein, the term “providing a prognosis” refers to providing a prediction of the probable course and outcome of preeclampsia. The methods can also be used to devise a suitable therapy for preeclampsia treatment, e.g., by indicating the severity or subtype of preeclampsia.

As used herein, the term “diagnosis” refers to detecting preeclampsia or a risk or propensity for development of preeclampsia. In any method of diagnosis exist false positives and false negatives. Any one method of diagnosis does not provide 100% accuracy.

PCR assays such as Taqman® assay available from Applied Biosystems can be used to analyze differential gene expression. In another embodiment, gas chromatography or mass spectroscopy can be used to detect the marker by analyzing either nucleic acid or protein. Any antibody-based technique for determining a level of expression of a protein of interest can be used to determine the biomarker. For example, immunoassays such as ELISA, Western blotting, flow cytometry, immunofluorescence, and immunohistochemistry can be used to detect protein in patient samples.

In one embodiment, a biopsy sample is obtained from a subject. Nucleic acid or protein is analyzed using mass spectroscopy techniques. In some cases, the analysis is automated.

Analysis of the biomarkers of the invention, using either protein or nucleic acid can be achieved, for example, by high pressure liquid chromatography (HPLC), alone or in combination with mass spectrometry (e.g., MALDI/MS, MALDI-TOF/IMS, tandem MS, etc.).

Analysis of the biomarkers of the invention can be achieved using routine techniques such as reverse-transcriptase polymerase chain reaction (RT-PCR), or any other methods based on hybridization to a nucleic acid sequence that is complementary to a portion of the marker coding sequence (e.g., slot blot hybridization) are also within the scope of the present invention. Applicable PCR amplification techniques are described in, e.g., Ausubel et al., Theophilus et al., and Innis et al., supra. General nucleic acid hybridization methods are described in Anderson, “Nucleic Acid Hybridization,” BIOS Scientific Publishers, 1999. Amplification or hybridization of a plurality of nucleic acid sequences (e.g., mRNA or cDNA) can also be performed from mRNA or cDNA sequences arranged in a microarray. Microarray methods are generally described in Hardiman, “Microarrays Methods and Applications: Nuts & Bolts,” DNA Press, 2003; and Baldi et al., “DNA Microarrays and Gene Expression: From Experiments to Data Analysis and Modeling,” Cambridge University Press, 2002.

Analysis of the nucleic acid marker can be performed using techniques known in the art including, without limitation, microarrays, polymerase chain reaction (PCR)-based analysis, sequence analysis, and electrophoretic analysis. A non-limiting example of a PCR-based analysis includes a Taqman® allelic discrimination assay available from Applied Biosystems. Non-limiting examples of electrophoretic analysis include slab gel electrophoresis such as agarose or polyacrylamide gel electrophoresis, capillary electrophoresis, and denaturing gradient gel electrophoresis.

Antibody reagents can be used in assays to detect expression levels of the biomarkers of the invention in patient samples using any of a number of immunoassays known to those skilled in the art. Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used. See, e.g., Self et al., Curr. Opin. Biotechnol., 7:60-65 (1996). The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. hnmunoassays can also be used in conjunction with laser induced fluorescence. See, e.g., Schmalzing et al., Electrophoresis, 18:2184-93 (1997); Bao, J. Chromatogr. B. Biomed. Sci., 699:463-80 (1997). Liposome immunoassays, such as flow-injection liposome immunoassays and liposome immunosensors, are also suitable for use in the present invention. See, e.g., Rongen et al., J. Immunol. Methods, 204:105-133 (1997). In addition, nephelometry assays, in which the formation of protein/antibody complexes results in increased light scatter that is converted to a peak rate signal as a function of the marker concentration, are suitable for use in the methods of the present invention. Nephelometry assays are commercially available from Beckman Coulter (Brea, Calif.; Kit #449430) and can be performed using a Behring Nephelometer Analyzer (Fink et al., J. Clin. Chem. Clin. Biochem., 27:261-276 (1989)).

Specific immunological binding of the antibody to nucleic acids can be detected directly or indirectly. Direct labels include fluorescent or luminescent tags, metals, dyes, radionuclides, and the like, attached to the antibody. An antibody labeled with iodine-125 (¹²⁵I) can be used. A chemiluminescence assay using a chemiluminescent antibody specific for the nucleic acid is suitable for sensitive, non-radioactive detection of protein levels. An antibody labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. An urease detection system can be used with a substrate such as urea-bromocresol purple (Sigma Immunochemicals; St. Louis, Mo.).

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

The antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), and the like. An assay strip can be prepared by coating the antibody or a plurality of antibodies in an array on a solid support. This strip can then be dipped into the test sample and processed quickly through washes and detection steps to generate a measurable signal, such as a colored spot.

A detectable moiety can be used in the assays described herein. A wide variety of detectable moieties can be used, with the choice of label depending on the sensitivity required, ease of conjugation with the antibody, stability requirements, and available instrumentation and disposal provisions. Suitable detectable moieties include, but are not limited to, radionuclides, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, and the like.

Useful physical formats comprise surfaces having a plurality of discrete, addressable locations for the detection of a plurality of different markers. Such formats include microarrays and certain capillary devices. See, e.g., Ng et al., J. Cell Mol. Med., 6:329-340 (2002); U.S. Pat. No. 6,019,944. In these embodiments, each discrete surface location may comprise antibodies or nucleic acid probes to immobilize one or more markers for detection at each location. Surfaces may alternatively comprise one or more discrete particles (e.g., microparticles or nanoparticles) immobilized at discrete locations of a surface, where the microparticles comprise antibodies to immobilize one or more markers for detection.

Analysis can be carried out in a variety of physical formats. For example, the use of microtiter plates or automation could be used to facilitate the processing of large numbers of test samples. Alternatively, single sample formats could be developed to facilitate diagnosis or prognosis in a timely fashion.

Alternatively, the antibodies or nucleic acid probes of the invention can be applied to sections of patient samples immobilized on microscope slides. The resulting antibody staining or in situ hybridization pattern can be visualized using any one of a variety of light or fluorescent microscopic methods known in the art.

### Compositioins, Kits and Integrated Systems

The invention provides compositions, kits and integrated systems for practicing the assays described herein using antibodies specific for the polypeptides or nucleic acids specific for the polynucleotides of the invention.

Kits for carrying out the diagnostic assays of the invention typically include a probe that comprises an antibody or nucleic acid sequence that specifically binds to polypeptides or polynucleotides of the invention, and a label for detecting the presence of the probe. The kits may include several antibodies or polynucleotide sequences encoding polypeptides of the invention, e.g., a cocktail of antibodies that recognize the proteins encoded by the biomarkers of the invention.

### Methods to Identify Compounds

A variety of methods may be used to identify compounds that prevent or treat preeclampsia. Typically, an assay that provides a readily measured parameter is adapted to be performed in the wells of multi-well plates in order to facilitate the screening of members of a library of test compounds as described herein. Thus, in one embodiment, an appropriate number of cells can be plated into the cells of a multi-well plate, and the effect of a test compound on the expression of a preeclampsia biomarker can be determined.

The compounds to be tested can be any small chemical compound, or a macromolecule, such as a protein, sugar, nucleic acid or lipid. Typically, test compounds will be small chemical molecules and peptides. Essentially any chemical compound can be used as a test compound in this aspect of the invention, although most often compounds that can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods are used which involve providing a combinatorial chemical or peptide library containing a large number of potential therapeutic compounds. Such “combinatorial chemical libraries” or “ligand libraries” are then screened in one or more assays, as described herein, to identify those library members (particular chemical species or subclasses) that display a desired characteristic activity. In this instance, such compounds are screened for their ability to reduce or increase the expression of the preeclampsia biomarkers of the invention.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries are well known to those of skill in the art. Such combinatorial chemical libraries include, but are not limited to, peptide libraries (see, e.g., U.S. Pat. No. 5,010,175, Furka, Int. J. Pept. Prot. Res., 37:487-493 (1991) and Houghton et al., Nature, 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., PNAS USA, 90:6909-6913 (1993)), vinylogous polypeptides (Hagihara et al., J. Amer. Chem. Soc., 114:6568 (1992)), nonpeptidal peptidomimetics with glucose scaffolding (Hirschmann et al., J. Amer. Chem. Soc., 114:9217-9218 (1992)), analogous organic syntheses of small compound libraries (Chen et al., J. Amer. Chem. Soc., 116:2661 (1994)), oligocarbamates (Cho et al., Science, 261:1303 (1993)), and/or peptidyl phosphonates (Campbell et al., J. Org. Chem., 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 96 modulators. If 1536 well plates are used, then a single plate can easily assay from about 100- about 1500 different compounds. It is possible to assay many plates per day; assay screens for up to about 6,000, 20,000, 50,000, or 100,000 or more different compounds is possible using the integrated systems of the invention.

Methods to Inhibit Marker Protein Expression using Nucleic Acids

A variety of nucleic acids, such as antisense nucleic acids, siRNAs or ribozymes, may be used to inhibit the function of the markers of this invention. Ribozymes that cleave mRNA at site-specific recognition sequences can be used to destroy target mRNAs, particularly through the use of hammerhead ribozymes. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art.

Gene targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of a target mRNA. In addition, ribozymes possess highly specific endoribonuclease activity, which autocatalytically cleaves the target sense mRNA.

With regard to antisense, siRNA or ribozyme oligonucleotides, phosphorothioate oligonucleotides can be used. Modifications of the phosphodiester linkage as well as of the heterocycle or the sugar may provide an increase in efficiency. Phophorothioate is used to modify the phosphodiester linkage. An N3′-P5′ phosphoramidate linkage has been described as stabilizing oligonucleotides to nucleases and increasing the binding to RNA. Peptide nucleic acid (PNA) linkage is a complete replacement of the ribose and phosphodiester backbone and is stable to nucleases, increases the binding affinity to RNA, and does not allow cleavage by RNAse H. Its basic structure is also amenable to modifications that may allow its optimization as an antisense component. With respect to modifications of the heterocycle, certain heterocycle modifications have proven to augment antisense effects without interfering with RNAse H activity. An example of such modification is C-5 thiazole modification. Finally, modification of the sugar may also be considered. 2′-O-propyl and 2′-methoxyethoxy ribose modifications stabilize oligonucleotides to nucleases in cell culture and in vivo.

Inhibitory oligonucleotides can be delivered to a cell by direct transfection or transfection and expression via an expression vector. Appropriate expression vectors include mammalian expression vectors and viral vectors, into which has been cloned an inhibitory oligonucleotide with the appropriate regulatory sequences including a promoter to result in expression of the antisense RNA in a host cell. Suitable promoters can be constitutive or development-specific promoters. Transfection delivery can be achieved by liposomal transfection reagents, known in the art (e.g., Xtreme transfection reagent, Roche, Alameda, Calif.; Lipofectamine formulations, Invitrogen, Carlsbad, Calif.). Delivery mediated by cationic liposomes, by retroviral vectors and direct delivery are efficient. Another possible delivery mode is targeting using antibody to cell surface markers for the target cells.

For transfection, a composition comprising one or more nucleic acid molecules (within or without vectors) can comprise a delivery vehicle, including liposomes, for administration to a subject, carriers and diluents and their salts, and/or can be present in pharmaceutically acceptable formulations. Methods for the delivery of nucleic acid molecules are described, for example, in Gilmore, et al., Curr Drug Delivery (2006) 3:147-5 and Patil, et al., AAPS Journal (2005) 7:E61-E77, each of which are incorporated herein by reference. Delivery of siRNA molecules is also described in several U.S. Patent Publications, including for example, 2006/0019912; 2006/0014289; 2005/0239687; 2005/0222064; and 2004/0204377, the disclosures of each of which are hereby incorporated herein by reference. Nucleic acid molecules can be administered to cells by a variety of methods known to those of skill in the art, including, but not restricted to, encapsulation in liposomes, by iontophoresis, by electroporation, or by incorporation into other vehicles, including biodegradable polymers, hydrogels, cyclodextrins (see,for example Gonzalez et al., 1999, Bioconjugate Chem., 10, 1068-1074; Wang et al., International PCT publication Nos. WO 03/47518 and WO 03/46185), poly(lactic-co-glycolic)acid (PLGA) and PLCA microspheres (see for example U.S. Pat. No. 6,447,796 and US Patent Application Publication No. 2002/130430), biodegradable nanocapsules, and bioadhesive microspheres, or by proteinaceous vectors (O'Hare and Normand, International PCT Publication No. WO 00/53722). In another embodiment, the nucleic acid molecules of the invention can also be formulated or complexed with polyethyleneimine and derivatives thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives.

Examples of liposomal transfection reagents of use with this invention include, for example: CellFectin, 1:1.5 (M/M) liposome formulation of the cationic lipid N,NI,NII,NIII-tetramethyl-N,NI,NII,NIII-tetrapalmit-y-spermine and dioleoyl phosphatidylethanolamine (DOPE) (GIBCO BRL); Cytofectin GSV, 2:1 (M/M) liposome formulation of a cationic lipid and DOPE (Glen Research); DOTAP (N-[1-(2,3-dioleoyloxy)-N,N,N-tri-methyl-ammoniummethylsulfate) (Boehringer Manheim); Lipofectamine, 3:1 (M/M) liposome formulation of the polycationic lipid DOSPA and the neutral lipid DOPE (GIBCO BRL); and (5) siPORT (Ambion); HiPerfect (Qiagen); X-treme GENE (Roche); RNAicarrier (Epoch Biolabs) and TransPass (New England Biolabs).

In some embodiments, antisense, siRNA, or ribozyme sequences are delivered into the cell via a mammalian expression vector. For example, mammalian expression vectors suitable for siRNA expression are commercially available, for example, from Ambion (e.g., pSilencer vectors), Austin, TX; Promega (e.g., GeneClip, siSTRIKE, SiLentGene), Madison, Wis.; Invitrogen, Carlsbad, Calif.; InvivoGen, San Diego, Calif.; and lmgenex, San Diego, Calif. Typically, expression vectors for transcribing siRNA molecules will have a U6 promoter.

In some embodiments, antisense, siRNA, or ribozyme sequences are delivered into cells via a viral expression vector. Viral vectors suitable for delivering such molecules to cells include adenoviral vectors, adeno-associated vectors, and retroviral vectors (including lentiviral vectors). For example, viral vectors developed for delivering and expressing siRNA oligonucleotides are commercially available from, for example, GeneDetect, Bradenton, Fla.; Ambion, Austin, Tex.; Invitrogen, Carlsbad, Calif.; Open BioSystems, Huntsville, Ala.; and Imgenex, San Diego, Calif.

### EXAMPLES

The following examples are offered to illustrate, but not to limit the claimed invention.

### Example 1

### Materials and Methods

### Tissue Collection

The University of California San Francisco Committee on Human Research approved the tissue procurement protocol. Informed consent was obtained from each parturient before delivery. Basal plate biopsy specimens of the maternal-fetal interface from preeclampsia patients and patients with preterm labor (control) were collected. The basal plate was dissected from the placenta proper, rinsed in PBS and diced into ˜3×3 mm³ pieces, which were snap-frozen in liquid nitrogen and stored at −70° C. All samples were processed and frozen within 1 h of delivery. For immunohistochemistry, biopsy samples of the basal plate were fixed in 3% paraformaldehyde in PBS (wt/vol), passed through a sucrose gradient (5-15% in PBS) and frozen in OCT (optimal cutting temperature).

In addition, biopsies of several regions of the tissue were also fixed in ten-percent neutral-buffered formalin and embedded in paraffin. Tissue sections prepared from the blocks were stained with hematoxylin and eosin and examined by using a light microscope. In all cases, normal morphological features were noted; there were no histological signs of placental or decidual pathology.

### Total RNA Extraction

RNA was isolated from snap-frozen basal plate specimens using a modified Trizol method that was developed during the course of this work (Haimov-Kochman, R. and Fisher, S. J. et al., Clin Chem, 52:159-160). Briefly, homogenization of 0.9-1 g of frozen basal plate specimens was carried out in 10 m l of cold Trizol reagent (Invitrogen, Frederick, Md.) on wet ice (0-4° C.). Cellular debris was pelleted by centrifugation at 12,000×g for 10 m in. Then the supernatant was transferred to Phase Lock Gel heavy tubes (Eppendorf, Germany), and RNA was isolated according to the manufacturer's instructions. The total RNA fraction was purified further by using an RNeasy mini kit (Qiagen) according to the manufacturer's instructions. Aliquots of the RNA isolated from the specimens were evaluated by using the Agilent RNA 6000 Nano LabChip kit (Agilent Technologies, Amstelveen, The Netherlands) on an Agilent Bioanalyzer 2100 system employing the nano assay for eukaryote total RNA. Capillary electrophoresis data in comma-separated value files were analyzed by using the Degradometer v. 1.41 software (available at www.dnaarrays.org) (Auer, H. and Lyianarachchi, S. et al., Nat Genet, 35:292-293). Only RNA with a degradation factor of <11 was used in subsequent microarray experiments.

### Microarray Hybridization

The microarray platform was the high-density HG-U133A and HG-U133B GeneChips (Affymetrix, Santa Clara, Calif.) that use 45,000-oligomer probe sets representing 39,000 transcripts. Hybridization was accomplished by using the protocol devised by the UCSF Gladstone (NHLBI) Genomics Core Facility (www.gladstone.ucsf. edu/gladstone/php/section). In brief, double-stranded cDNAs were generated from total RNA samples by using SuperScript II reverse transcriptase (Invitrogen) and a T7-oligo primer (Qiagen). Biotin-labeled cRNA was synthesized by in vitro transcription using an Enzo Bioassay RNA labeling kit (Enzo Diagnostics, Farmingdale, N.Y.). The labeled cRNA was purified with an RNeasy column (Qiagen). Before hybridization, the quality of all in vitro transcription products was evaluated by using the Agilent Bioanalyzer 2100 system. Then the cRNA was fragmented at 94° C. for 35 min in buffer (Tris-acetate 40 mmol/L, potassium acetate 100 mmol/L, magnesium acetate 30 mmol/L, pH 8.1). Samples from individual basal plates were analyzed separately. Specifically, the HG-U133A and HG-U133B Affymetrix GeneChips were each hybridized with 15 μg of cRNA, and then washed, stained and imaged at the Gladstone Genomics Core Facility by using standard Affymetrix protocols. Data files were deposited in the GEO (Gene Expression Omnibus) data repository with accession # *.

### Data Analysis

The raw image data were analyzed by using GeneChip Expression Analysis software (Affymetrix) to produce perfect match and mismatch values. Subsequently, quality control, preprocessing, and linear modeling were performed using Bioconductor (Gentleman, R. C. and Carey, V. J. et al., Genome Biol, 5:R80), an open-source and open-development software project based on the R statistical package (www.r-project.org). Clustering analysis was performed using Acuity software (Molecular Devices Corp., Sunnyvale, Calif.). Initial hybridization quality was assessed by using Bioconductor package affyPLM, and the slight variations in quality were compensated for during the preprocessing stage, which was performed in two steps. First, we used a Probe Level robust linear model (Bolstad, B. M., Dissertation, University of California, Berkeley (2004)) to obtain separate normalized log intensities for each chip (i.e., background subtraction, quantile normalization, and probe set summarization). Second, we applied a global median normalization at the probe set level to all A and B GeneChips (n=72) and then combined these data into a matrix of log₂-based gene expression measures, in which columns corresponded to different cRNA samples, and rows corresponded to the different probe sets.

Then differentially expressed genes were selected by determining the log odds ratio with significance set at B>0 The normalized intensity values for this data set were centered to the median intensity value for each probe set, after which the probe sets were ranked according to their M values (representing fold change) and depicted as a gene expression color map.

### Pathway and Network Analysis

Initially, gene ontogeny (GO) annotations were determined (www.genetools.microarray.ntnu.no) and used to categorize the differentially expressed genes according to the biological processes in which they were involved (level 2). When biological process information was lacking, genes were annotated according to molecular function. To determine if there was a significant overrepresentation of differentially expressed genes in particular functions or physiologic processes, the data set was analyzed by using Ingenuity Pathway Analysis 3.1 software (www.Ingenuity.com). The data set containing gene identifiers and their corresponding expression values was uploaded as an Excel spreadsheet using the template provided in the application. Each gene identifier was mapped to its corresponding gene object in the Ingenuity Pathways Knowledge Base. Differentially regulated genes, identified by using an adjusted P-value of <0.05 as the cut off, were then used as the starting point for generating biological networks.

### Quantitative PCR

Reverse transcription of basal plate (total) RNA was carried out by using the TaqMan Gold RT-PCR kit (Applied Biosystems, Foster City, Calif.) as described by the manufacturer, followed by real-time PCR, performed in triplicate by using the Applied Biosystems 9700HT sequence detection system. All templates were amplified by using Assay-on-Demand kits (Applied Biosystems) or primer/probe sets designed by the UCSF Biomolecular Research Center (see Supplemental Data [Table SI]). Briefly, 5 μL of cDNA was added to 20 μL of 1×TaqMan Universal PCR Master Mix containing AmpErase UNG and 1 μL of a primer/probe. Negative controls contained RNA that was either not reverse transcribed or lacked template inputs. Reactions were incubated at 50° C. for 2 min, then 95° C. for 10 m in, followed by 40 cycles of 95° C. for 15 s and 60° C. for 1 min. Relative quantification was determined by using the standard curve method (see ABI User Bulletin #2; www.appledbiosystems.com). In preliminary experiments, we investigated the utility of 11 potential targets as endogenous controls (endogenous control plate, Applied Biosystems). The results showed that the 18S rRNA did not vary with gestational age. Accordingly, the levels of this transcript were used to obtain normalized values for the target amplicons. Then, these values were calibrated to a 24-wk control sample, the earliest gestational age included in our analysis. Results were reported as the relative fold mRNA levels ±SD for each basal plate specimen. The means of the preterm labor contols and preterm preeclampsia samples were compared using a two-tailed Student's t-test (P<0.05).

### Immunohistochemistry

Frozen sections (5 μM) cut from OCT-embedded tissues were washed in PBS and nonspecific reactivity was blocked with 3% BSA, 0.1% Triton X-100, 0.5% Tween-20 in PBS for 30 min. Then the experimental sections were incubated with an antibody of interest for 1 h, after which they were washed in PBS three times for 5 min. Negative controls were incubated in the absence of the primary antibody. Then, both experimental and control sections were incubated in rat anti-human cytokeratin (CK) (1:100; 7D3 (Damsky, C. H. and Fitzgerald, M. L. et al., J. Clin Invest, 89:210-222)]) for 1 h and washed in PBS as described above. To localize the bound primary antibodies, the sections were incubated with Alexa Fluor 594-conjugated goat anti-mouse IgG (1:1,000; Molecular Probes Inc., Eugene, Oreg.) and fluorescein isothiocyanate-labeled donkey anti-rat IgG (1:200; Jackson ImmunoResearch Laboratories, West Grove, Pa.) antibodies for 30 min and washed again in PBS. Tissue sections were mounted in Vectashield containing 4′-6-diamidino-2-phenylindol (Vector Laboratories, Burlingame, Calif.), which allowed visualization of the nuclei. Immunoreactivity was imaged using a Leica DM 5000B fluorescent microscope equipped with a Leica DFC 350FX digital camera (Leica Instruments, San Jose, Calif.).

### Protein Function Annotation by Sequence Homology and Structural Similarity

To determine the function of the differentially expressed genes that lacked annotations (www.genetools.microarray.ntnu.no; July, 2005), we used protein sequence homology searches along with protein structure modeling. Briefly, protein sequences for the differentially expressed genes were extracted by using their UniGene identifiers (Wheeler, D. L. and Barrett, T. et al., Nucleic Acids Res, 34:D 173-180). Homology searches were done using PSI-BLAST (Altschul, S. F. and Madden, T. L. et al., Nucleic Acids Res, 25:3389-3402). Five iterations of the PSI-BLAST were run using an e-value cutoff of 10-5 for sequences to be included in the profile. For protein sequences with detectable homology to other proteins of known structure, comparative structure models were built through the MODWEB server (Eswar, N. and John, B. et al., Nucleic Acids Res, 31:3375-3380), which uses the program MODELLER (Sali, A. and Blundell, T. L., J Mol Biol, 234:779-815 (1993)). Proteins that could not be assigned a function based on homology searches were subjected to threading using the mGenTHREADER software (McGuffin, L. J. and Jones, D. T., Bioinformatics, 19:874-881 (2003)).

### Example 2

### Differentially Expressed Genes in the Maternal-Fetal Interface in Preeclampsia Study Design

Using the general methods described above, we undertook to identify genes that were differentially expressed at the maternal-fetal interface (basal plate) in subjects with preeclampsia versus subjects experiencing preterm labor as controls. The characteristics of the study design are shown below in Table 1. A total of 23 samples were analyzed. 11 of these were preterm labor (PTL) controls and 12 were samples from preeclampsia pateients with the clinical presentations shown in Table 1, including an elevated blood pressure of ±140/90 and proteinuria greater than 300 mg/24 hours or a protein dipstick reading of ≧1+.

TABLE 1
Study Design
Prospectively collect human basal plate
Preterm pregnancies (24-36 wk)
Singleton
Preeclampsia:
Elevated BP ± 140/90
Proteinuria > 300 mg/24 h or ≧1+ clean catch
No PPROM, infection or maternal disease (except HTN)
Controls:
Preterm labor
no PPROM, infection or maternal disease
Create tissue bank of frozen and fixed tissue

| Diagnosis | Number of Samples |
|---|---|
| PTL | 11 |
| Preeclampsia | 12 |
| TOTAL | 23 |

### Clinical Characteristics of Study Groups

Some of the clinical characteristics of the patients from which samples were taken is shown below in Table 2. Further characteristics of the preeclampsia group are shown in Table 3.

TABLE 2
Clinical Characteristics of Study Groups

| | | | p |
|---|---|---|---|
| | PTL (n = 11) | PE (n = 12) | value |
| Maternal Age (yr) | 30.2 ± 7.1 | 30.7 ± 9.1 | ns |
| Gestational Age (wks) | 31.0 ± 4.6 | 32.1 ± 3.3 | ns |
| Nulliparus | 7 (64%) | 9 (75%) | ns |
| C/S | 2 (18%) | 6 (50%) | ns |
| Labored | 11 (100%) | 10 (83%) | ns |

TABLE 3
Characteristics of Preeclampsia Group (n = 12)

| | |
|---|---|
| Severe PE | 12 (100%) |
| Severe by BP | 9 (83%) |
| Severe by proteinuria | 6 (50%) |
| Severe by abnormal lab | 2 (17%) |
| Creatinine (median) | 0.9 [0.5-1.3] |
| Ecclampsia | 2 (17%) |
| Superimposed PE | 2 (17%) |
| Fetal Abnormality* | 5 (42%) |

*IUGR, oligohydramnios or abnormal dopplers

### Differentially Expressed Genes in Preeclampsia

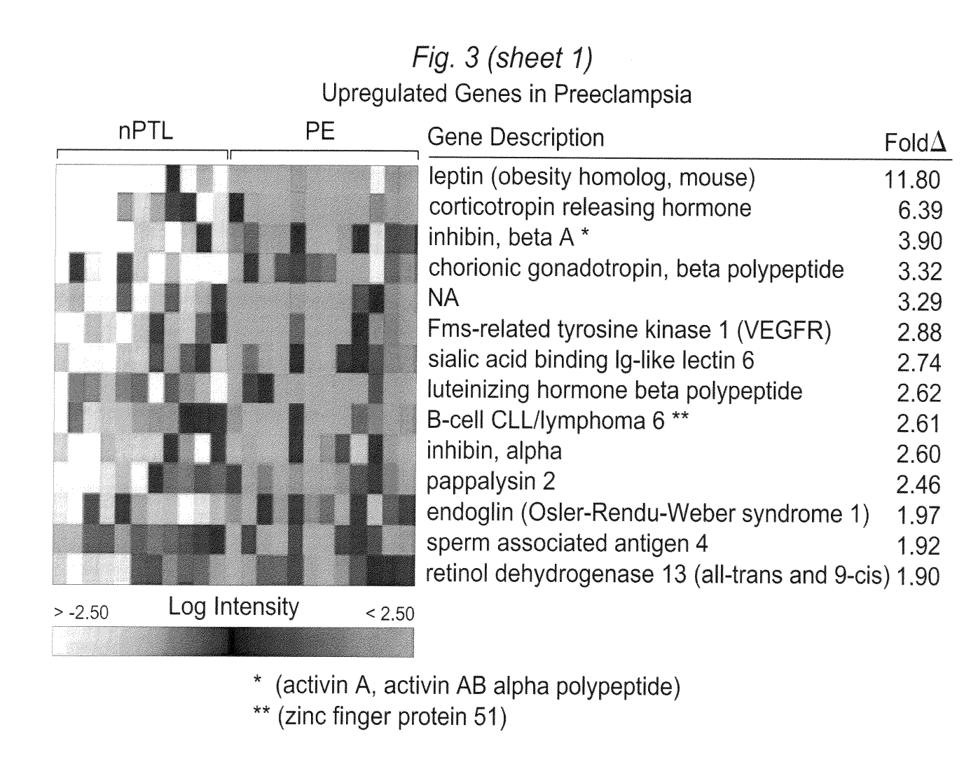

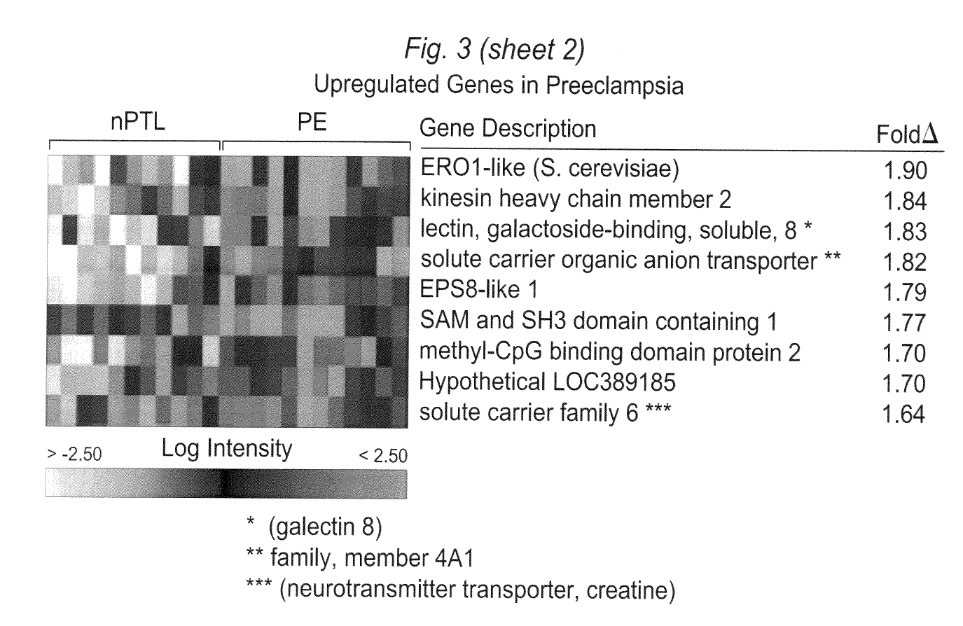

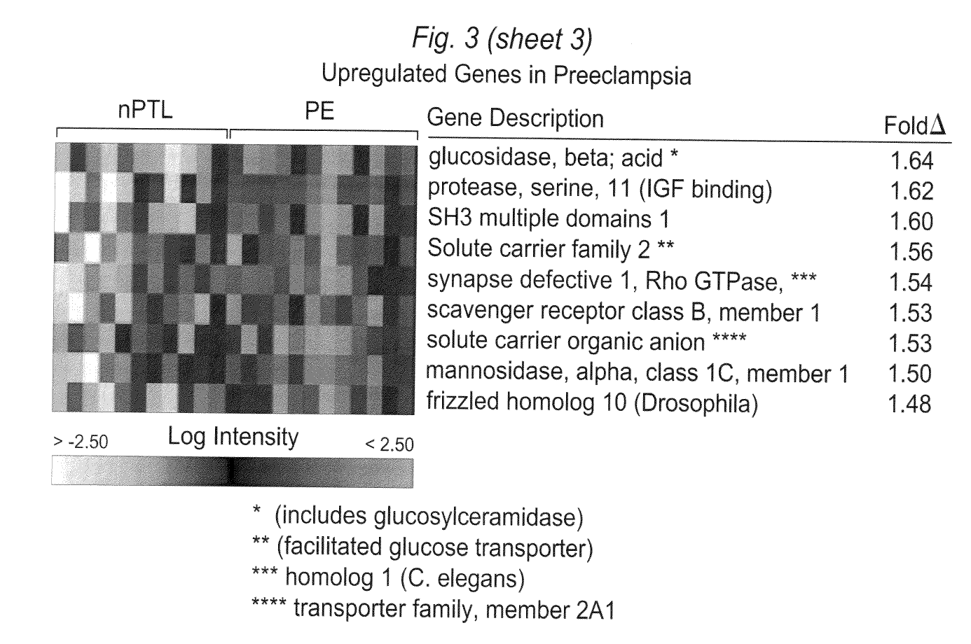

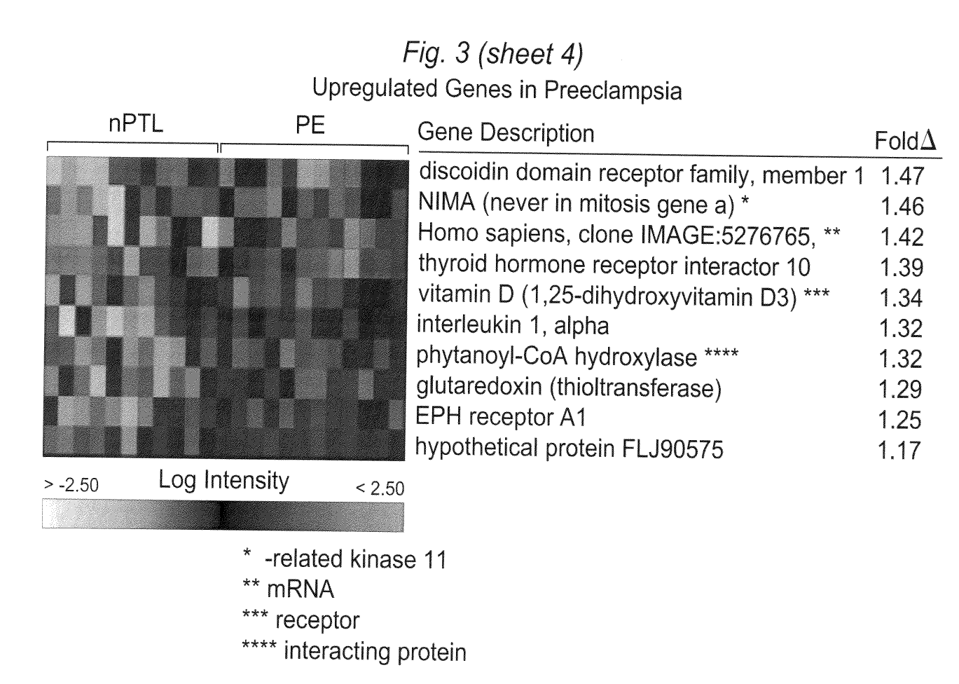

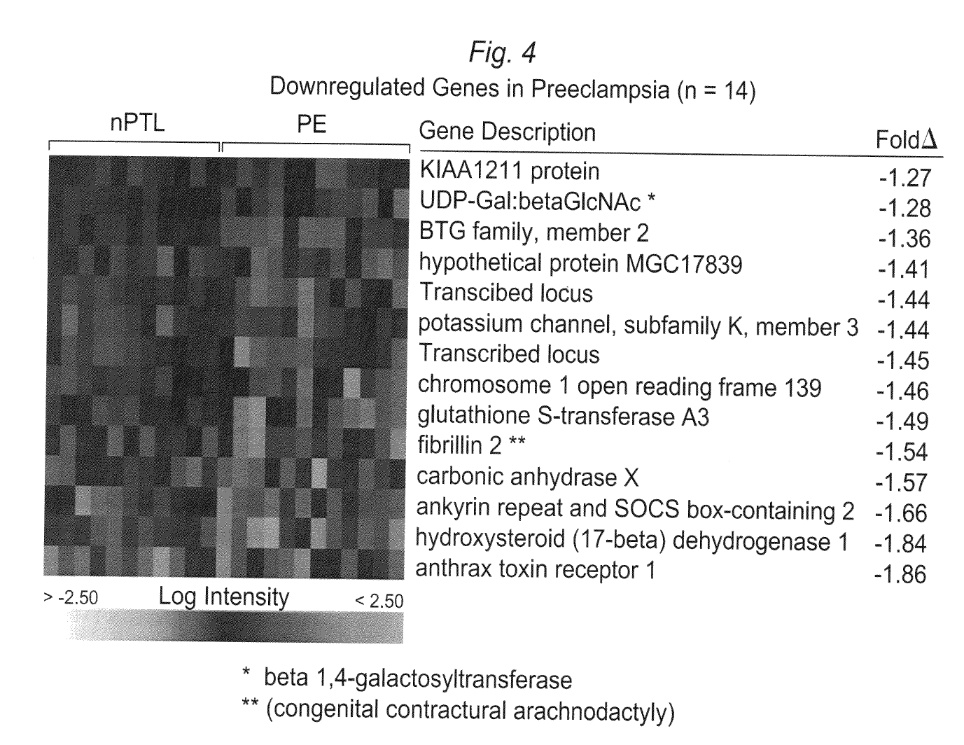

Applying the methods described above, we compared the gene expression profiles of basal plate samples from patients diagnosed with preeclampsia with samples from preterm labor individuals, which served as a control group. As a result of these experiments, we identified 65 genes that are differentially expressed in preeclampsia (see FIGS. 3 and 4). Nine of the DNA sequences identified were non-annotated or encoded hypothetical proteins. As shown in FIG. 3, the extent of upregulation ranged from 11.8 fold for leptin to 1.17 for a hypothetical protein. The down regulated genes as shown in FIG. 4, showed a 1.27 to 1.86 fold reduction in expression levels. Two of the upregulated genes, leptin and Fms-related tyrosine kinase (also known VEGF receptor 1 and the parent molecule of sFlt-1) were previously known to be upregulated in preeclampsia, and thus, establish the validity of this approach for identifying genes that are differentially regulated in preeclampsia.

The GenBank accession numbers for the genes found to be up or down regulated in this invention include those shown in Table 4 below.

TABLE 4
Genes up regulated or down regulated in preeclampsia

| ProbesetId | Symbol | M | B | FDR | A | Entrez. Gene | Unigene | Name |
|---|---|---|---|---|---|---|---|---|
| 207092_at | LEP | 4.317 | 10.22 | 0 | 9.343 | 3952 | Hs.194236 | leptin (obesity |
| | | | | | | | | homolog, mouse) |
| 205629_s_at | CRH | 2.967 | 8.015 | 0 | 10.139 | 1392 | Hs.75294 | corticotropin |
| | | | | | | | | releasing hormone |
| 203548_s_at | LPL | 2.325 | 1.546 | 0.051 | 8.702 | 4023 | Hs.180878 | lipoprotein lipase |
| 210511_s_at | INHBA | 2.198 | 6.623 | 0.001 | 11.763 | 3624 | Hs.28792 | inhibin, beta A |
| | | | | | | | | (activin A, activin |
| | | | | | | | | AB alpha |
| | | | | | | | | polypeptide) |
| 203549_s_at | LPL | 2.139 | 1.209 | 0.062 | 9.554 | 4023 | Hs.180878 | lipoprotein lipase |
| 221200_at | — | 1.978 | 5.467 | 0.003 | 10.346 | — | — | — |
| 205630_at | CRH | 1.938 | 3.392 | 0.012 | 12.741 | 1392 | Hs.75294 | corticotropin |
| | | | | | | | | releasing hormone |
| 222033_s_at | FLT1 | 1.92 | 7.827 | 0 | 8.654 | 2321 | Hs.507621 | Fms-related |
| | | | | | | | | tyrosine kinase 1 |
| | | | | | | | | (vascular |
| | | | | | | | | endothelial growth |
| | | | | | | | | factor/vascular |
| | | | | | | | | permeability factor |
| | | | | | | | | receptor) |
| 205387_s_at | CGB /// | 1.709 | 0.509 | 0.102 | 10.469 | 1082 /// | Hs.446683 | chorionic |
| | CGB5 /// | | | | | 93659 /// | | gonadotropin, beta |
| | CGB7 | | | | | 94027 | | polypeptide /// |
| | | | | | | | | chorionic |
| | | | | | | | | gonadotropin, beta |
| | | | | | | | | polypeptide 5 /// |
| | | | | | | | | chorionic |
| | | | | | | | | gonadotropin, beta |
| | | | | | | | | polypeptide 7 |
| 203980_at | FABP4 | 1.69 | 0.384 | 0.112 | 9.051 | 2167 | Hs.391561 | fatty acid binding |
| | | | | | | | | protein 4, |
| | | | | | | | | adipocyte |
| 203140_at | BCL6 | 1.642 | 8.377 | 0 | 8.649 | 604 | Hs.478588 | B-cell |
| | | | | | | | | CLL/lymphoma 6 |
| | | | | | | | | (zinc finger protein |
| | | | | | | | | 51) /// B-cell |
| | | | | | | | | CLL/lymphoma 6 |
| | | | | | | | | (zinc finger protein |
| | | | | | | | | 51) |
| 204926_at | INHBA | 1.616 | 5.29 | 0.003 | 6.147 | 3624 | Hs.28792 | inhibin, beta A |
| | | | | | | | | (activin A, activin |
| | | | | | | | | AB alpha |
| | | | | | | | | polypeptide) |
| 227140_at | INHBA | 1.604 | 1.371 | 0.056 | 11.429 | 3624 | Hs.28792 | Inhibin, beta A |
| | | | | | | | | (activin A, activin |
| | | | | | | | | AB alpha |
| | | | | | | | | polypeptide) |
| 210287_s_at | FLT1 | 1.576 | 1.646 | 0.047 | 8.908 | 2321 | Hs.507621 | fms-related |
| | | | | | | | | tyrosine kinase 1 |
| | | | | | | | | (vascular |
| | | | | | | | | endothelial growth |
| | | | | | | | | factor/vascular |
| | | | | | | | | permeability factor |
| | | | | | | | | receptor) |
| 210796_x_at | SIGLEC6 | 1.575 | 4.126 | 0.006 | 8.848 | 946 | Hs.397255 | sialic acid binding |
| | | | | | | | | Ig-like lectin 6 |
| 206520_x_at | — | 1.457 | 4.006 | 0.007 | 9.445 | — | — | — |
| 206519_x_at | — | 1.423 | 1.23 | 0.061 | 6.353 | — | — | — |
| 228237_at | PAPPA2 | 1.418 | 2.398 | 0.026 | 8.61 | 60676 | Hs.187284 | pappalysin 2 |
| 214471_x_at | LHB | 1.393 | 1.996 | 0.035 | 8.148 | 3972 | Hs.154704 | luteinizing |
| | | | | | | | | hormone beta |
| | | | | | | | | polypeptide |
| 210141_s_at | INHA | 1.363 | 2.423 | 0.026 | 8.404 | 3623 | Hs.407506 | inhibin, alpha |
| 203592_s_at | FSTL3 | 1.292 | 1.277 | 0.06 | 10.157 | 10272 | Hs.529038 | follistatin-like 3 |
| | | | | | | | | (secreted |
| | | | | | | | | glycoprotein) |
| 209122_at | ADFP | 1.272 | 1.294 | 0.06 | 11.532 | 123 | Hs.3416 | adipose |
| | | | | | | | | differentiation- |
| | | | | | | | | related protein |
| 210665_at | TFPI | 1.203 | 2.379 | 0.026 | 8.74 | 7035 | Hs.516578 | tissue factor |
| | | | | | | | | pathway inhibitor |
| | | | | | | | | (lipoprotein- |
| | | | | | | | | associated |
| | | | | | | | | coagulation |
| | | | | | | | | inhibitor) |
| 211918_x_at | PAPPA2 | 1.197 | 0.642 | 0.095 | 10.305 | 60676 | Hs.187284 | pappalysin 2 /// |
| | | | | | | | | pappalysin 2 |
| 222646_s_at | ERO1L | 1.181 | 2.372 | 0.026 | 7.384 | 30001 | Hs.525339 | ERO1-like (S. cerevisiae) |
| 226497_s_at | FLT1 | 1.152 | 1.873 | 0.04 | 10.815 | 2321 | Hs.507621 | Fms-related |
| | | | | | | | | tyrosine kinase 1 |
| | | | | | | | | (vascular |
| | | | | | | | | endothelial growth |
| | | | | | | | | factor/vascular |
| | | | | | | | | permeability factor |
| | | | | | | | | receptor) |
| 201809_s_at | ENG | 1.137 | 6.754 | 0.001 | 10.008 | 2022 | Hs.76753 | endoglin (Osler- |
| | | | | | | | | Rendu-Weber |
| | | | | | | | | syndrome 1) |
| 203434_s_at | MME | 1.123 | 1.781 | 0.042 | 8.696 | 4311 | Hs.307734 | membrane metallo- |
| | | | | | | | | endopeptidase |
| | | | | | | | | (neutral |
| | | | | | | | | endopeptidase, |
| | | | | | | | | enkephalinase, |
| | | | | | | | | CALLA, CD10) |
| 200653_s_at | CALM1 | 1.099 | 3.819 | 0.008 | 10.03 | 801 | Hs.282410 | calmodulin 1 |
| | | | | | | | | (phosphorylase |
| | | | | | | | | kinase, delta) |
| 212328_at | KIAA1102 | 1.097 | 2.057 | 0.033 | 8.646 | 22998 | Hs.335163 | KIAA1102 protein |
| 202018_s_at | LTF | 1.085 | 0.413 | 0.11 | 6.41 | 4057 | Hs.529517 | lactotransferrin |
| 225467_s_at | RDH13 | 1.082 | 3.376 | 0.012 | 9.5 | 112724 | Hs.327631 | retinol |
| | | | | | | | | dehydrogenase 13 |
| | | | | | | | | (all-trans and 9-cis) |
| 214397_at | MBD2 | 1.07 | 1.204 | 0.062 | 5.952 | 8932 | Hs.25674 | methyl-CpG |
| | | | | | | | | binding domain |
| | | | | | | | | protein 2 |
| 226022_at | SASH1 | 1.058 | 1.434 | 0.055 | 9.651 | 23328 | Hs.193133 | SAM and SH3 |
| | | | | | | | | domain containing 1 |
| 226498_at | FLT1 | 1.048 | 1.418 | 0.055 | 11.649 | 2321 | Hs.507621 | Fms-related |
| | | | | | | | | tyrosine kinase 1 |
| | | | | | | | | (vascular |
| | | | | | | | | endothelial growth |
| | | | | | | | | factor/vascular |
| | | | | | | | | permeability factor |
| | | | | | | | | receptor) |
| 203087_s_at | KIF2 | 1.048 | 4.729 | 0.004 | 9.417 | 3796 | Hs.552575 | kinesin heavy |
| | | | | | | | | chain member 2 |
| 203407_at | PPL | 1.037 | 2.105 | 0.032 | 7.801 | 5493 | Hs.192233 | periplakin |
| 200632_s_at | NDRG1 | 1.017 | 1.702 | 0.045 | 9.792 | 10397 | Hs.372914 | N-myc downstream |
| | | | | | | | | regulated gene 1 |
| 219888_at | SPAG4 | 1.012 | 5.166 | 0.003 | 6.365 | 6676 | Hs.123159 | sperm associated |
| | | | | | | | | antigen 4 |
| 212327_at | KIAA1102 | 1.003 | 1.294 | 0.06 | 9.223 | 22998 | Hs.335163 | KIAA1102 protein |
| 227919_at | — | 1.002 | 0.72 | 0.093 | 9.323 | — | Hs.515223 | Homo sapiens, |
| | | | | | | | | Similar to unnamed |
| | | | | | | | | HERV-H protein, |
| | | | | | | | | clone |
| | | | | | | | | IMAGE: 3996038, |
| | | | | | | | | mRNA |
| 228758_at | — | 0.988 | 3.392 | 0.012 | 7.766 | 389185 | Hs.478589 | Hypothetical |
| | | | | | | | | LOC389185 |
| 213236_at | SASH1 | 0.977 | 5.878 | 0.002 | 9.215 | 23328 | Hs.193133 | SAM and SH3 |
| | | | | | | | | domain containing 1 |
| 203476_at | TPBG | 0.965 | 3.668 | 0.01 | 10.073 | 7162 | Hs.82128 | trophoblast |
| | | | | | | | | glycoprotein |
| 214396_s_at | MBD2 | 0.951 | 3.586 | 0.01 | 7.026 | 8932 | Hs.25674 | methyl-CpG |
| | | | | | | | | binding domain |
| | | | | | | | | protein 2 |
| 219911_s_at | SLCO4A1 | 0.928 | 1.567 | 0.051 | 8.318 | 28231 | Hs.235782 | solute carrier |
| | | | | | | | | organic anion |
| | | | | | | | | transporter family, |
| | | | | | | | | member 4A1 |
| 212873_at | HA-1 | 0.927 | 2.969 | 0.016 | 8.387 | 23526 | Hs.465521 | minor |
| | | | | | | | | histocompatibility |
| | | | | | | | | antigen HA-1 |
| 210732_s_at | LGALS8 | 0.919 | 1.522 | 0.052 | 6.489 | 3964 | Hs.4082 | lectin, galactoside- |
| | | | | | | | | binding, soluble, 8 |
| | | | | | | | | (galectin 8) |
| 228434_at | BTNL9 | 0.909 | 0.321 | 0.114 | 5.116 | 153579 | Hs.546502 | butyrophilin-like 9 |
| 221665_s_at | EPS8L1 | 0.9 | 4.89 | 0.004 | 9.091 | 54869 | Hs.438862 | EPS8-like 1 |
| 210664_s_at | TFPI | 0.898 | 0.465 | 0.105 | 10.256 | 7035 | Hs.516578 | tissue factor |
| | | | | | | | | pathway inhibitor |
| | | | | | | | | (lipoprotein- |
| | | | | | | | | associated |
| | | | | | | | | coagulation |
| | | | | | | | | inhibitor) |
| 220456_at | C20orf38 | 0.892 | 0.964 | 0.08 | 6.524 | 55304 | Hs.272242 | chromosome 20 |
| | | | | | | | | open reading frame |
| | | | | | | | | 38 |
| 209682_at | CBLB | 0.889 | 0.92 | 0.083 | 8.287 | 868 | Hs.430589 | Cas-Br-M (murine) |
| | | | | | | | | ecotropic retroviral |
| | | | | | | | | transforming |
| | | | | | | | | sequence b |
| 215990_s_at | BCL6 | 0.883 | 2.529 | 0.024 | 7.086 | 604 | Hs.478588 | B-cell |
| | | | | | | | | CLL/lymphoma 6 |
| | | | | | | | | (zinc finger protein |
| | | | | | | | | 51) |
| 209581_at | HRASLS3 | 0.869 | 0.618 | 0.095 | 9.38 | 11145 | Hs.502775 | HRAS-like |
| | | | | | | | | suppressor 3 |
| 209563_x_at | CALM1 | 0.867 | 0.717 | 0.093 | 11.033 | 801 | Hs.282410 | calmodulin 1 |
| | | | | | | | | (phosphorylase |
| | | | | | | | | kinase, delta) |
| 201811_x_at | SH3BP5 | 0.842 | 0.292 | 0.116 | 9.633 | 9467 | Hs.257761 | SH3-domain |
| | | | | | | | | binding protein 5 |
| | | | | | | | | (BTK-associated) |
| 236518_at | KIAA1984 | 0.833 | 0.338 | 0.114 | 6.091 | 84960 | Hs.370555 | KIAA1984 |
| 210589_s_at | GBA /// | 0.825 | 4.599 | 0.005 | 8.946 | 2629 /// | Hs.511984 | glucosidase, beta; |
| | GBAP | | | | | 2630 | | acid (includes |
| | | | | | | | | glucosylceramidase) |
| | | | | | | | | /// glucosidase, |
| | | | | | | | | beta; acid, |
| | | | | | | | | pseudogene |
| 91826_at | EPS8L1 | 0.823 | 4.293 | 0.006 | 10.259 | 54869 | Hs.438862 | EPS8-like 1 |
| 218779_x_at | EPS8L1 | 0.818 | 2.575 | 0.023 | 10.25 | 54869 | Hs.438862 | EPS8-like 1 |
| 224817_at | SH3MD1 | 0.798 | 3.12 | 0.015 | 9.512 | 9644 | Hs.159368 | SH3 multiple |
| | | | | | | | | domains 1 |
| 221655_x_at | EPS8L1 | 0.788 | 3.074 | 0.015 | 9.964 | 54869 | Hs.43886 | EPS8-like 1 |
| 41644_at | SASH1 | 0.787 | 4.558 | 0.005 | 10.115 | 23328 | Hs.193133 | SAM and SH3 |
| | | | | | | | | domain containing 1 |
| 211984_at | CALM1 | 0.782 | 1.714 | 0.045 | 9.658 | 801 | Hs.282410 | calmodulin 1 |
| | | | | | | | | (phosphorylase |
| | | | | | | | | kinase, delta) |
| 218507_at | HIG2 | 0.773 | 0.67 | 0.094 | 9.357 | 29923 | Hs.521171 | hypoxia-inducible |
| | | | | | | | | protein 2 |
| 215812_s_at | SLC6A8 | 0.767 | 5.337 | 0.003 | 8.256 | 386757 /// | Hs.540696 | solute carrier |
| | /// | | | | | 6535 | | family 6 |
| | FLJ43855 | | | | | | | (neurotransmitter |
| | | | | | | | | transporter, |
| | | | | | | | | creatine), member |
| | | | | | | | | 8 /// similar to |
| | | | | | | | | sodium- and |
| | | | | | | | | chloride-dependent |
| | | | | | | | | creatine transporter |
| 218816_at | LRRC1 | 0.764 | 1.25 | 0.061 | 8.739 | 55227 | Hs.485581 | leucine rich repeat |
| | | | | | | | | containing 1 |
| 40016_g_at | MAST4 | 0.75 | 0.682 | 0.094 | 8.725 | 23227 | Hs.133539 | microtubule |
| | | | | | | | | associated |
| | | | | | | | | serine/threonine |
| | | | | | | | | kinase family |
| | | | | | | | | member 4 |
| 219764_at | FZD10 | 0.749 | 2.134 | 0.032 | 6.843 | 11211 | Hs.31664 | frizzled homolog |
| | | | | | | | | 10 (Drosophila) |
| 213332_at | PAPPA2 | 0.746 | 5.043 | 0.003 | 13.356 | 60676 | Hs.187284 | Pappalysin 2 |
| 204368_at | SLCO2A1 | 0.739 | 4.123 | 0.006 | 10.332 | 6578 | Hs.518270 | solute carrier |
| | | | | | | | | organic anion |
| | | | | | | | | transporter family, |
| | | | | | | | | member 2A1 |
| 214268_s_at | MTMR4 | 0.733 | 0.86 | 0.086 | 10.027 | 9110 | Hs.514373 | myotubularin |
| | | | | | | | | related protein 4 |
| 213598_at | HSA9761 | 0.728 | 2.99 | 0.016 | 8.14 | 27292 | Hs.533222 | Dimethyladenosine |
| | | | | | | | | transferase |
| 44702_at | 7h3 | 0.727 | 5.342 | 0.003 | 9.997 | 85360 | Hs.528701 | hypothetical |
| | | | | | | | | protein FLJ13511 |
| 201185_at | PRSS11 | 0.725 | 2.95 | 0.016 | 12.783 | 5654 | Hs.501280 | protease, serine, 11 |
| | | | | | | | | (IGF binding) |
| 226459_at | PIK3AP1 | 0.72 | 0.199 | 0.125 | 9.383 | 118788 | Hs.310456 | phosphoinositide- |
| | | | | | | | | 3-kinase adaptor |
| | | | | | | | | protein 1 |
| 209093_s_at | GBA /// | 0.711 | 2.256 | 0.029 | 8.683 | 2629 /// | Hs.511984 | glucosidase, beta; |
| | GBAP | | | | | 2630 | | acid (includes |
| | | | | | | | | glucosylceramidase) |
| | | | | | | | | /// glucosidase, |
| | | | | | | | | beta; acid, |
| | | | | | | | | pseudogene |
| 220794_at | GREM2 | 0.699 | 2.191 | 0.031 | 5.835 | 64388 | Hs.98206 | gremlin 2 homolog, |
| | | | | | | | | cysteine knot |
| | | | | | | | | superfamily |
| | | | | | | | | (Xenopus laevis) |
| 208934_s_at | LGALS8 | 0.69 | 1.414 | 0.055 | 8.473 | 3964 | Hs.4082 | lectin, galactoside- |
| | | | | | | | | binding, soluble, 8 |
| | | | | | | | | (galectin 8) |
| 218918_at | MAN1C1 | 0.689 | 0.482 | 0.104 | 11.671 | 57134 | Hs.197043 | mannosidase, |
| | | | | | | | | alpha, class 1C, |
| | | | | | | | | member 1 |
| 214180_at | — | 0.689 | 4.23 | 0.006 | 9.262 | — | Hs.546727 | MRNA; cDNA |
| | | | | | | | | DKFZp564H203 |
| | | | | | | | | (from clone |
| | | | | | | | | DKFZp564H203) |
| 230710_at | — | 0.687 | 0.405 | 0.11 | 7.003 | — | Hs.446388 | CDNA FLJ41489 |
| | | | | | | | | fis, clone |
| | | | | | | | | BRTHA2004582 |
| 240055_at | SLC2A14 | 0.685 | 2.969 | 0.016 | 6.138 | 144195 | Hs.210227 | Solute carrier |
| | | | | | | | | family 2 |
| | | | | | | | | (facilitated glucose |
| | | | | | | | | transporter), |
| | | | | | | | | member 3 |
| 212242_at | TUBA1 | 0.684 | 0.136 | 0.131 | 7.13 | 7277 | Hs.75318 | tubulin, alpha 1 |
| | | | | | | | | (testis specific) |
| 228740_at | — | 0.676 | 3.388 | 0.012 | 5.46 | — | Hs.26766 | Homo sapiens, |
| | | | | | | | | clone |
| | | | | | | | | IMAGE: 5276765, |
| | | | | | | | | mRNA |
| 219542_at | NEK11 | 0.675 | 4.243 | 0.006 | 7.705 | 79858 | Hs.200813 | NIMA (never in |
| | | | | | | | | mitosis gene a)- |
| | | | | | | | | related kinase 11 |
| 208959_s_at | TXNDC4 | 0.668 | 0.502 | 0.102 | 10.019 | 23071 | Hs.154023 | thioredoxin domain |
| | | | | | | | | containing 4 |
| | | | | | | | | (endoplasmic |
| | | | | | | | | reticulum) |
| 203575_at | CSNK2A2 | 0.665 | 0.306 | 0.115 | 7.385 | 1459 | Hs.82201 | casein kinase 2, |
| | | | | | | | | alpha prime |
| | | | | | | | | polypeptide |
| 209343_at | EFHD1 | 0.658 | 2.351 | 0.026 | 11.866 | 80303 | Hs.516767 | EF hand domain |
| | | | | | | | | family, member D1 |
| 219424_at | EBI3 | 0.657 | 0 | 0.143 | 11.93 | 10148 | Hs.501452 | Epstein-Barr virus |
| | | | | | | | | induced gene 3 |
| 203086_at | KIF2 | 0.653 | 0.632 | 0.095 | 6.749 | 3796 | Hs.552575 | Kinesin heavy |
| | | | | | | | | chain member 2 |
| 201041_s_at | DUSP1 | 0.64 | 0.13 | 0.131 | 11.892 | 1843 | Hs.171695 | dual specificity |
| | | | | | | | | phosphatase 1 |
| 201819_at | SCARB1 | 0.637 | 0.802 | 0.09 | 9.179 | 949 | Hs.298813 | scavenger receptor |
| | | | | | | | | class B, member 1 |
| 213790_at | ADAM12 | 0.626 | 0.344 | 0.114 | 11.267 | 8038 | Hs.386283 | A disintegrin and |
| | | | | | | | | metalloproteinase |
| | | | | | | | | domain 12 (meltrin |
| | | | | | | | | alpha) |
| 225750_at | ERO1L | 0.622 | 0.162 | 0.129 | 8.979 | 30001 | Hs.525339 | ERO1-like (S. cerevisiae) |
| 232649_at | COLM | 0.604 | 0.087 | 0.136 | 7.772 | 342035 | Hs.526441 | Collomin |
| 44783_s_at | HEY1 | 0.581 | 0.265 | 0.119 | 8.461 | 23462 | Hs.234434 | hairy/enhancer-of- |
| | | | | | | | | split related with |
| | | | | | | | | YRPW motif 1 |
| 225911_at | LOC255743 | 0.562 | 1.416 | 0.055 | 6.019 | 255743 | Hs.518921 | hypothetical |
| | | | | | | | | protein |
| | | | | | | | | LOC255743 |
| 202255_s_at | SIPA1L1 | 0.486 | 0.007 | 0.143 | 8.038 | 26037 | Hs.208846 | signal-induced |
| | | | | | | | | proliferation- |
| | | | | | | | | associated 1 like 1 |
| 204254_s_at | VDR | 0.475 | 3.037 | 0.016 | 5.731 | 7421 | Hs.524368 | vitamin D (1,25- |
| | | | | | | | | dihydroxyvitamin |
| | | | | | | | | D3) receptor |
| 240113_at | SASH1 | 0.473 | 1.816 | 0.042 | 6.696 | 23328 | Hs.193133 | SAM and SH3 |
| | | | | | | | | domain containing 1 |
| 244444_at | PKD1L2 | 0.473 | 0.34 | 0.114 | 5.838 | 114780 | Hs.413525 | polycystic kidney |
| | | | | | | | | disease 1-like 2 |
| 206662_at | GLRX | 0.449 | 3.207 | 0.014 | 11.092 | 2745 | Hs.28988 | glutaredoxin |
| | | | | | | | | (thioltransferase) |
| 236090_at | — | 0.443 | 0.084 | 0.136 | 7.033 | — | Hs.529962 | Transcribed locus |
| 1007_s_at | DDR1 | 0.442 | 0.761 | 0.091 | 10.027 | 780 | Hs.520004 | discoidin domain |
| | | | | | | | | receptor family, |
| | | | | | | | | member 1 |
| 208200_at | — | 0.433 | 0.84 | 0.087 | 6.601 | — | — | — |
| 223874_at | ARP3BETA | 0.431 | 0.707 | 0.093 | 6.721 | 57180 | Hs.490655 | actin-related |
| | | | | | | | | protein 3-beta |
| 204391_x_at | TIF1 | 0.427 | 0.779 | 0.091 | 7.266 | 8805 | Hs.490287 | transcriptional |
| | | | | | | | | intermediary factor 1 |
| 207790_at | LRRC1 | 0.426 | 0.257 | 0.119 | 6.654 | 55227 | Hs.485581 | leucine rich repeat |
| | | | | | | | | containing 1 |
| 202952_s_at | ADAM12 | 0.42 | 0.516 | 0.102 | 12.576 | 8038 | Hs.386283 | a disintegrin and |
| | | | | | | | | metalloproteinase |
| | | | | | | | | domain 12 (meltrin |
| | | | | | | | | alpha) |
| 205866_at | FCN3 | 0.415 | 0.57 | 0.099 | 6.839 | 8547 | Hs.333383 | ficolin |
| | | | | | | | | (collagen/fibrinogen |
| | | | | | | | | domain |
| | | | | | | | | containing) 3 |
| | | | | | | | | (Hakata antigen) |
| 205325_at | PHYHIP | 0.41 | 1.384 | 0.056 | 6.319 | 9796 | Hs.334688 | phytanoyl-CoA |
| | | | | | | | | hydroxylase |
| | | | | | | | | interacting protein |
| 203833_s_at | TGOLN2 | 0.399 | 0.764 | 0.091 | 7.624 | 10618 | Hs.14894 | trans-golgi network |
| | | | | | | | | protein 2 |
| 202308_at | SREBF1 | 0.386 | 0.005 | 0.143 | 8.68 | 6720 | Hs.190284 | sterol regulatory |
| | | | | | | | | element binding |
| | | | | | | | | transcription factor 1 |
| 218972_at | TTC17 | 0.378 | 0.208 | 0.125 | 8.553 | 55761 | Hs.191186 | tetratricopeptide |
| | | | | | | | | repeat domain 17 |
| 221989_at | RPL10 | 0.373 | 0.327 | 0.114 | 8.773 | 6134 | Hs.401929 | ribosomal protein |
| | | | | | | | | L10 |
| 204328_at | EVER1 | 0.329 | 0.712 | 0.093 | 7.674 | 11322 | Hs.16165 | epidermodysplasia |
| | | | | | | | | verruciformis 1 |
| 241372_at | ZC3HDC6 | 0.293 | 0.17 | 0.128 | 5.45 | 376940 | Hs.190477 | zinc finger CCCH |
| | | | | | | | | type domain |
| | | | | | | | | containing 6 |
| 230186_at | MGC17839 | −0.282 | 1.246 | 0.061 | 7.681 | 219902 | Hs.380228 | hypothetical |
| | | | | | | | | protein MGC17839 |
| 218345_at | HCA112 | −0.284 | 0.62 | 0.095 | 8.59 | 55365 | Hs.12126 | hepatocellular |
| | | | | | | | | carcinoma- |
| | | | | | | | | associated antigen |
| | | | | | | | | 112 |
| 229664_at | MAPK8 | −0.332 | 0.631 | 0.095 | 6.245 | 5599 | Hs.522924 | Mitogen-activated |
| | | | | | | | | protein kinase 8 |
| 227231_at | KIAA1211 | −0.358 | 0.682 | 0.094 | 3.913 | 57482 | Hs.479783 | KIAA1211 protein |
| 235638_at | RASSF6 | −0.4 | 0.058 | 0.138 | 4.749 | 166824 | Hs.529677 | Ras association |
| | | | | | | | | (RalGDS/AF-6) |
| | | | | | | | | domain family 6 |
| 226048_at | MAPK8 | −0.429 | 0.57 | 0.099 | 7.468 | 5599 | Hs.522924 | mitogen-activated |
| | | | | | | | | protein kinase 8 |
| 229944_at | — | −0.447 | 0.138 | 0.131 | 4.087 | — | Hs.106795 | CDNA FLJ13136 |
| | | | | | | | | fis, clone |
| | | | | | | | | NT2RP3003139 |
| 201236_s_at | BTG2 | −0.484 | 2.57 | 0.023 | 10.051 | 7832 | Hs.519162 | BTG family, |
| | | | | | | | | member 2 |
| 229634_at | FLJ90586 | −0.493 | 0.551 | 0.1 | 7.14 | 135932 | Hs.17558 | hypothetical |
| | | | | | | | | protein FLJ90586 |
| 224963_at | SLC26A2 | −0.538 | 0.022 | 0.143 | 8.145 | 1836 | Hs.302738 | solute carrier |
| | | | | | | | | family 26 (sulfate |
| | | | | | | | | transporter), |
| | | | | | | | | member 2 |
| 231029_at | — | −0.544 | 0.336 | 0.114 | 6.121 | — | Hs.436057 | Transcribed locus |
| 238497_at | MGC17839 | −0.554 | 1.49 | 0.053 | 6.221 | 219902 | Hs.380228 | hypothetical |
| | | | | | | | | protein MGC17839 |
| 228950_s_at | FLJ23091 | −0.564 | 0.52 | 0.102 | 8.238 | 79971 | Hs.479491 | putative NFkB |
| | | | | | | | | activating protein |
| | | | | | | | | 373 |
| 222102_at | GSTA3 | −0.593 | 0.894 | 0.083 | 8.943 | 2940 | Hs.102484 | glutathione S- |
| | | | | | | | | transferase A3 |
| 218717_s_at | LEPREL1 | −0.595 | 1.078 | 0.07 | 7.749 | 55214 | Hs.374191 | leprecan-like 1 |
| 203184_at | FBN2 | −0.629 | 0.352 | 0.114 | 10.612 | 2201 | Hs.519294 | fibrillin 2 |
| | | | | | | | | (congenital |
| | | | | | | | | contractural |
| | | | | | | | | arachnodactyly) |
| 227230_s_at | KIAA1211 | −0.682 | 0.895 | 0.083 | 6.43 | 57482 | Hs.479783 | KIAA1211 protein |
| 227915_at | ASB2 | −0.803 | 1.783 | 0.042 | 6.434 | 51676 | Hs.510327 | ankyrin repeat and |
| | | | | | | | | SOCS box- |
| | | | | | | | | containing 2 |
| 222549_at | CLDN1 | −0.827 | 2.106 | 0.032 | 7.946 | 9076 | Hs.439060 | claudin 1 |
| 220092_s_at | ANTXR1 | −0.882 | 0.071 | 0.137 | 5.479 | 84168 | Hs.165859 | anthrax toxin |
| | | | | | | | | receptor 1 |
| 205829_at | HSD17B1 | −1.05 | 6.648 | 0.001 | 11.032 | 3292 | Hs.500159 | hydroxysteroid |
| | | | | | | | | (17-beta) |
| | | | | | | | | dehydrogenase 1 |
| 202734_at | TRIP10 | 0.47 | 2.57 | 0.03 | 7.95 | 9322 | Hs.515094 | thyroid hormone |
| | | | | | | | | receptor interactor |
| | | | | | | | | 10 |
| 208200_at | IL1A | 0.4 | 0.71 | 0.14 | 6.56 | 3552 | Hs.1722 | interleukin 1, alpha |
| 204637_at | CGA | 0.21 | 0.01 | 0.24 | 14.12 | 1081 | Hs.119689 | glycoprotein |
| | | | | | | | | hormones, alpha |
| | | | | | | | | polypeptide |
| 44702_at | SYDE1 | 0.63 | 2.01 | 0.04 | 9.94 | 85360 | Hs.528701 | synapse defective |
| | | | | | | | | 1, Rho GTPase, |
| | | | | | | | | homolog 1 |
| | | | | | | | | (C. elegans) |
| 205977_s_at | EPHA1 | 0.33 | 0.04 | 0.22 | 7.95 | 2041 | Hs.89839 | EPH receptor A1 |
| 238682_at | FLJ90575 | 0.23 | 0.44 | 0.17 | 6.38 | 257236 | Hs.381181 | hypothetical |
| | | | | | | | | protein FLJ90575 |
| 221485_at | B4GALT5 | −0.35 | 0.24 | 0.2 | 7.99 | 9334 | Hs.370487 | UDP- |
| | | | | | | | | Gal:betaGlcNAc |
| | | | | | | | | beta 1,4- |
| | | | | | | | | galactosyltransferase, |
| | | | | | | | | polypeptide 5 |
| 237134_at | — | −0.53 | 1.86 | 0.05 | 5.4 | | Hs.26920 | transcribed locus |
| 205952_at | KCNK3 | −0.53 | 0.01 | 0.22 | 7.04 | 3777 | Hs.24040 | potassium channel, |
| | | | | | | | | subfamily K, |
| | | | | | | | | member 3 |
| 220889_s_at | CA10 | 0.65 | 0.15 | 0.22 | 6.08 | 56934 | Hs.463466 | carbonic anhydrase X |
| 236201_at | — | 0.874 | 0.222 | 0.206 | | 8.97 | Hs.93739 | Transcribed locus |
| 205891_at | ADORA2B | −0.349 | 0.002 | 0.243 | 6.948 | 136 | Hs.167046 | adenosine A2b |
| | | | | | | | | receptor |
| 204580_at | MMP12 | −1.135 | −3.195 | 1 | 8.878 | 4321 | Hs.1695 | matrix |
| | | | | | | | | metalloproteinase |
| | | | | | | | | 12 (macrophage |
| | | | | | | | | elastase) |
| 220191_at | GKN1 | −1.203 | −1.555 | 0.577 | 6.829 | 56287 | Hs.69319 | gastrokine 1 |
| 228950_s_at | C1orf139 | −0.54 | 0.38 | 0.18 | 8.18 | 79971 | Hs.479491 | chromosome 1 |
| | | | | | | | | open reading frame |
| | | | | | | | | 139 |

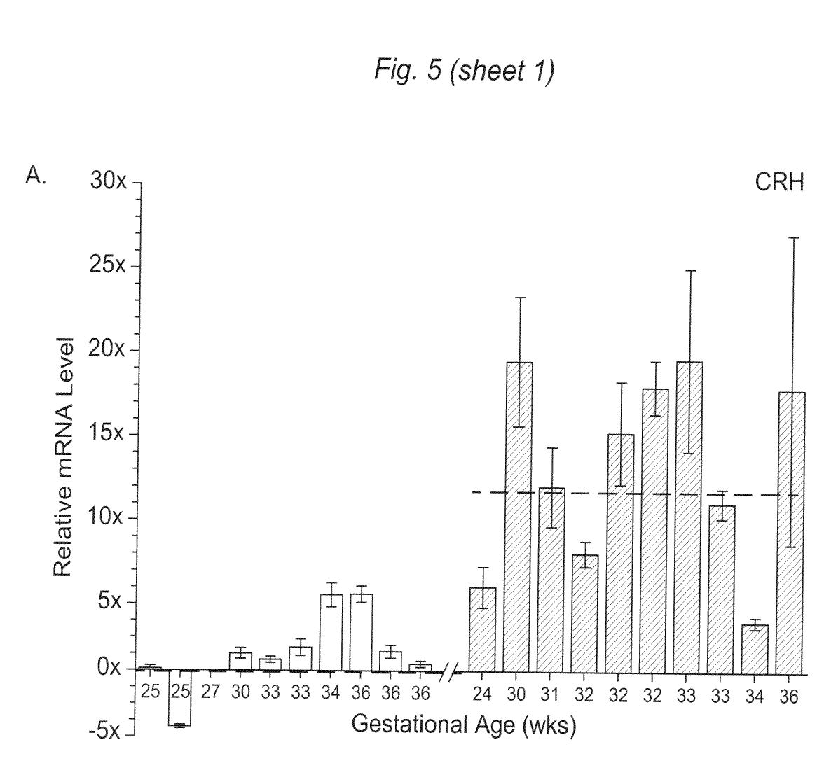

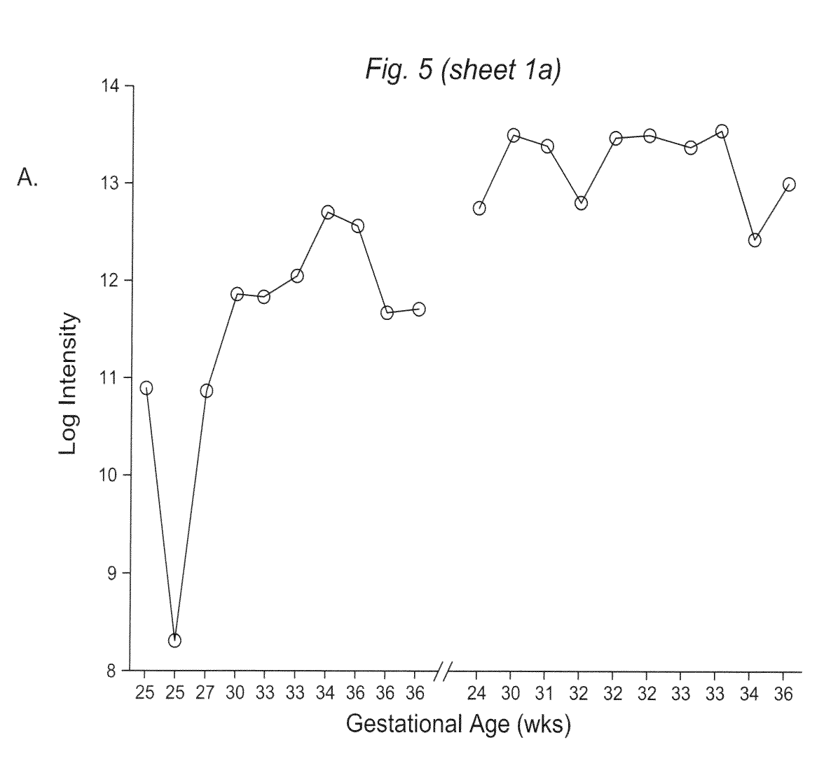

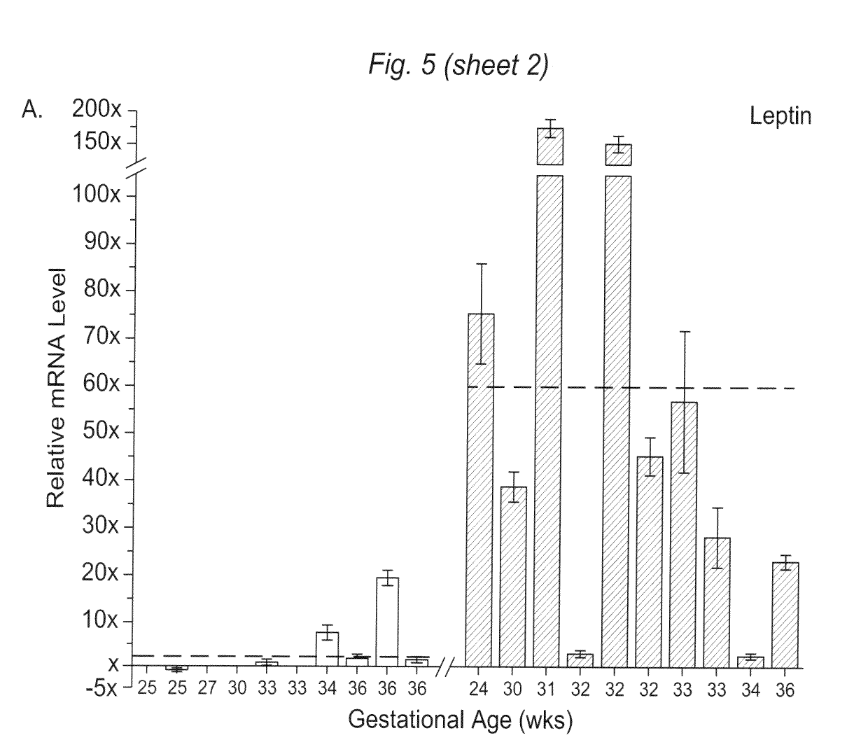

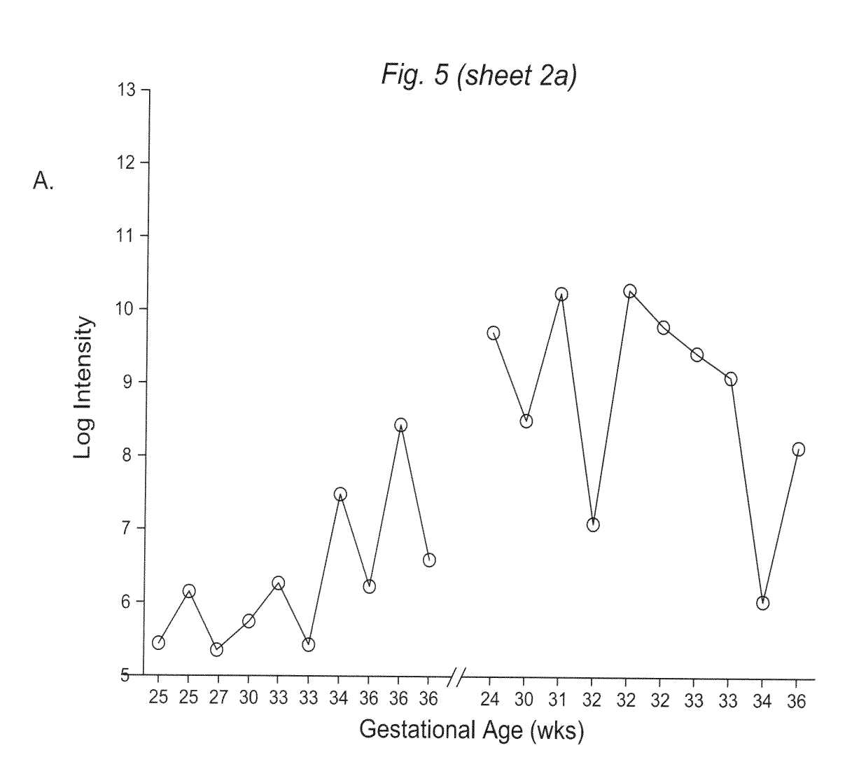

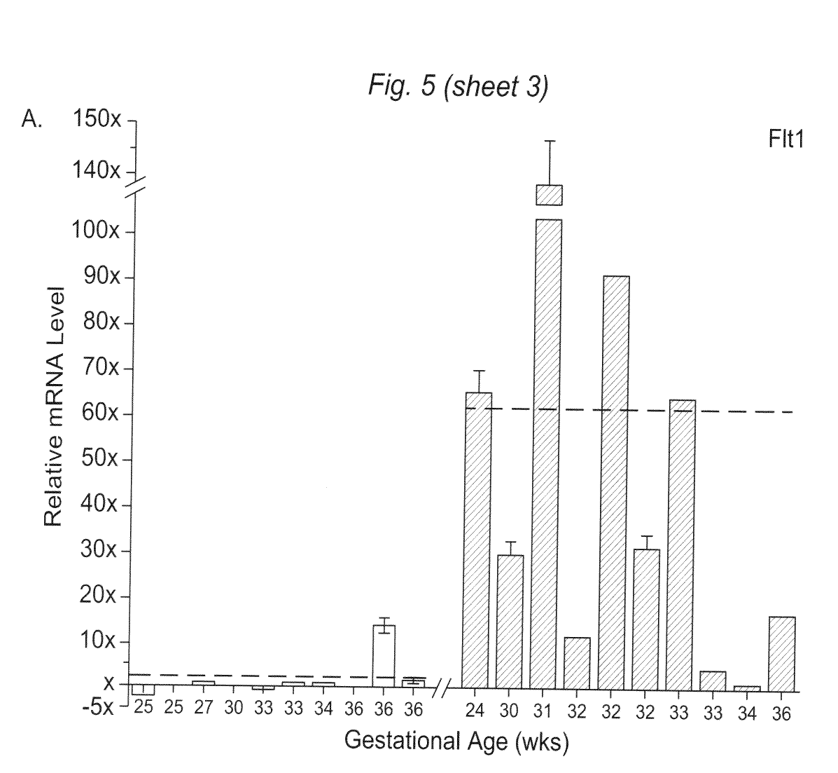

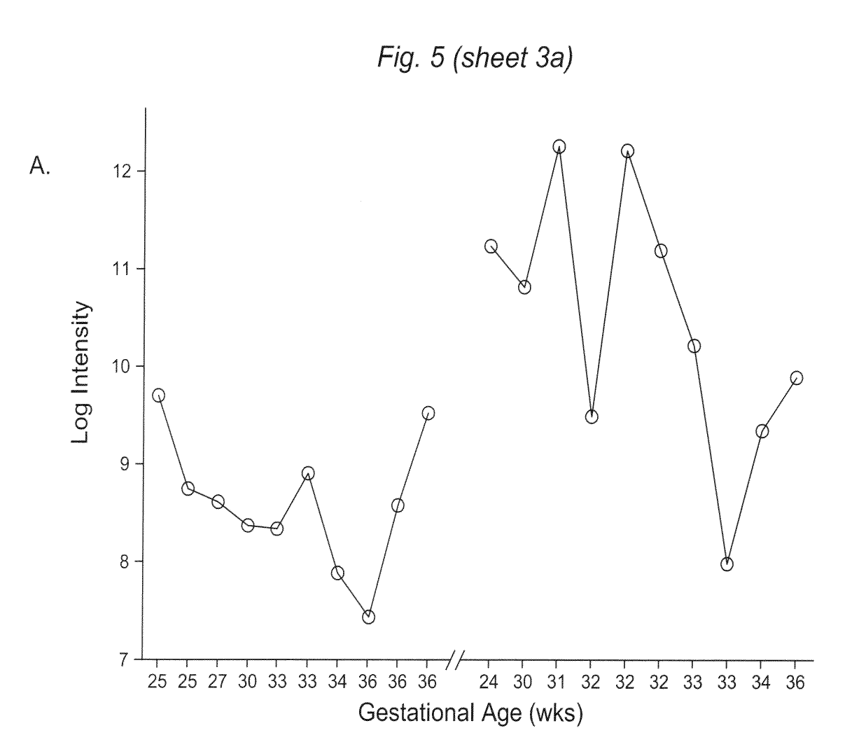

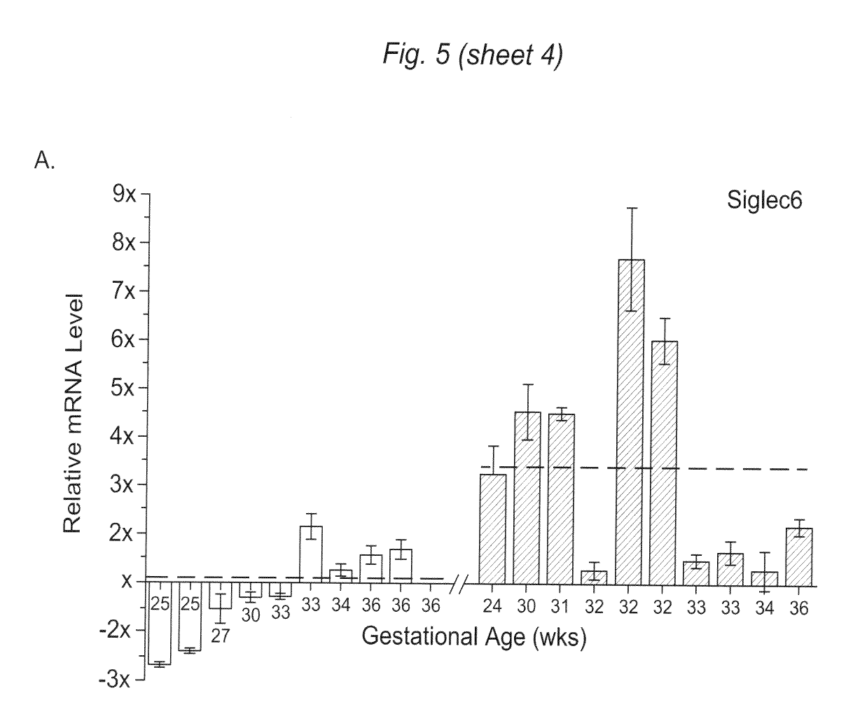

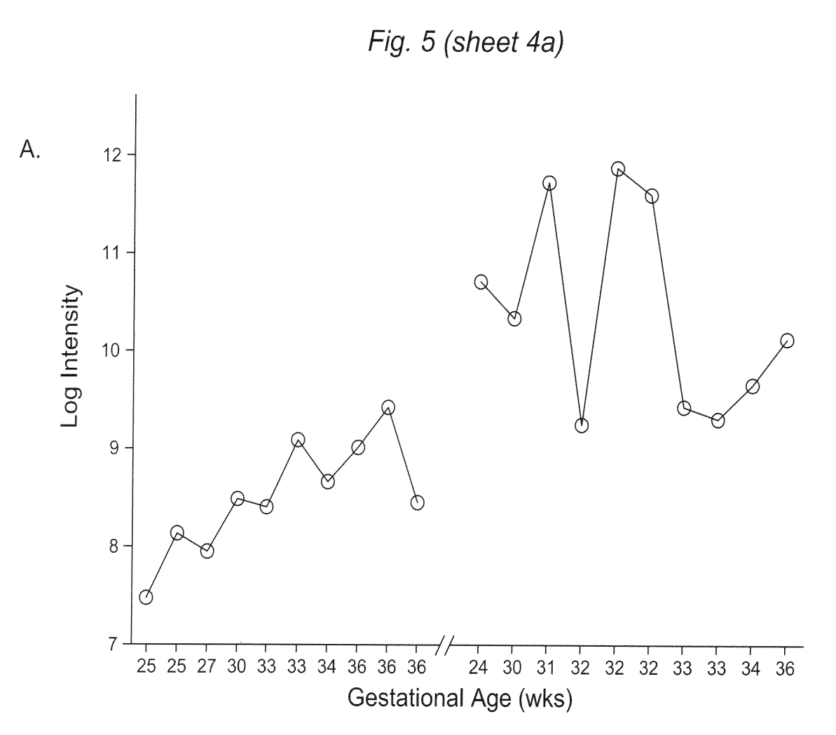

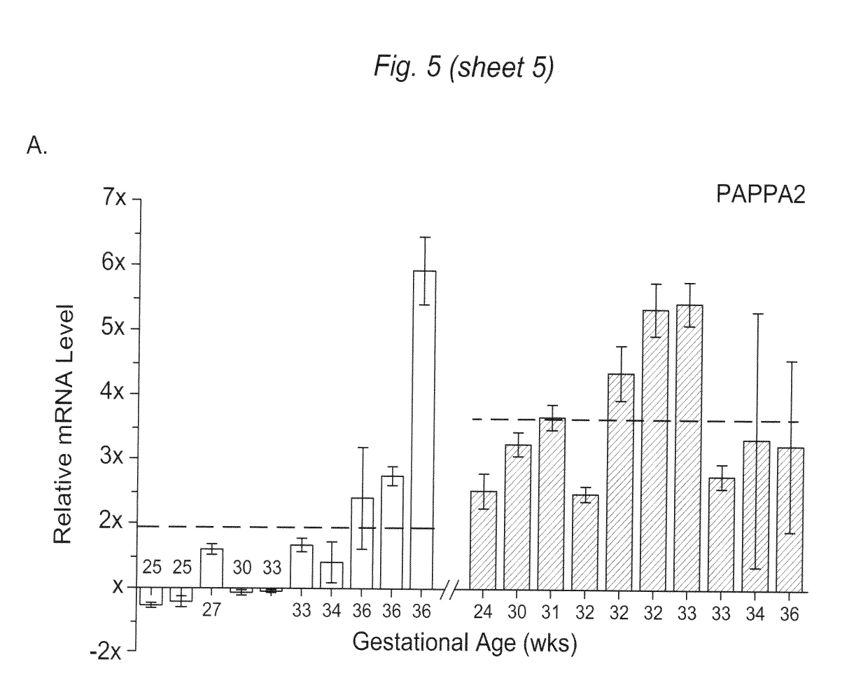

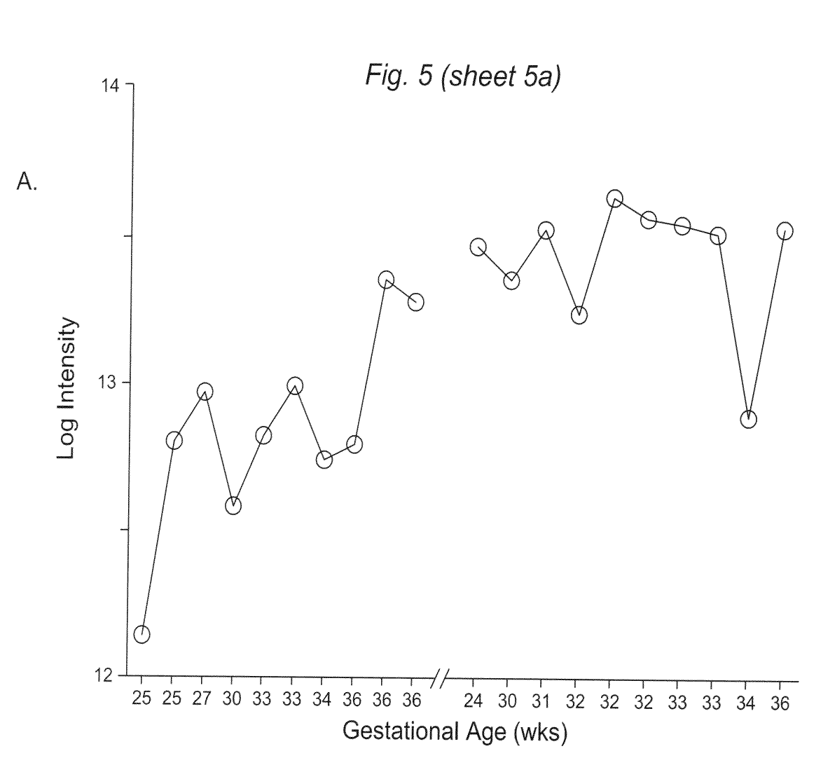

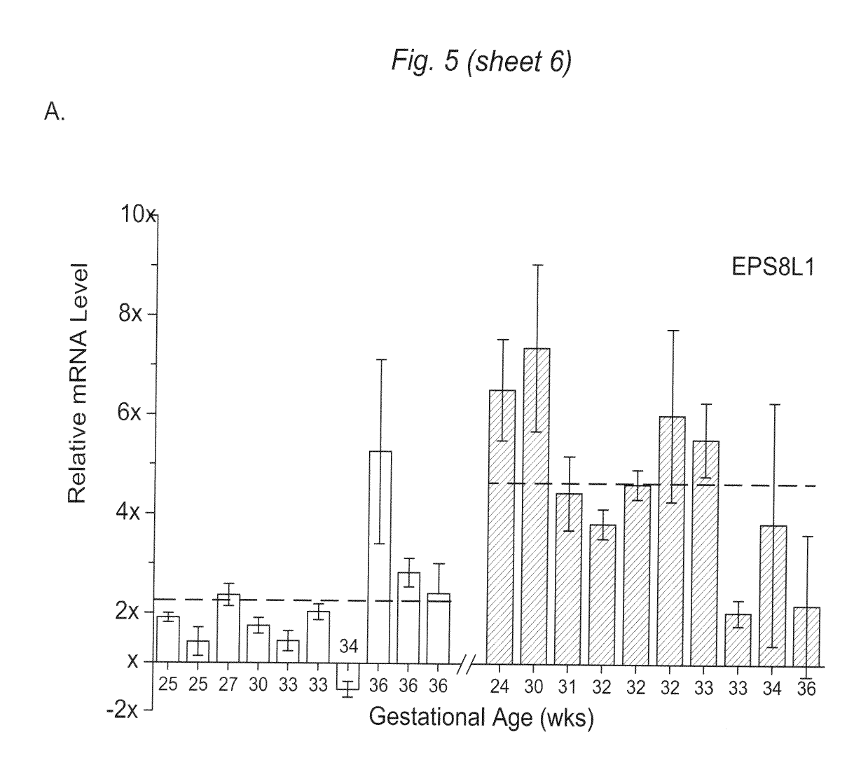

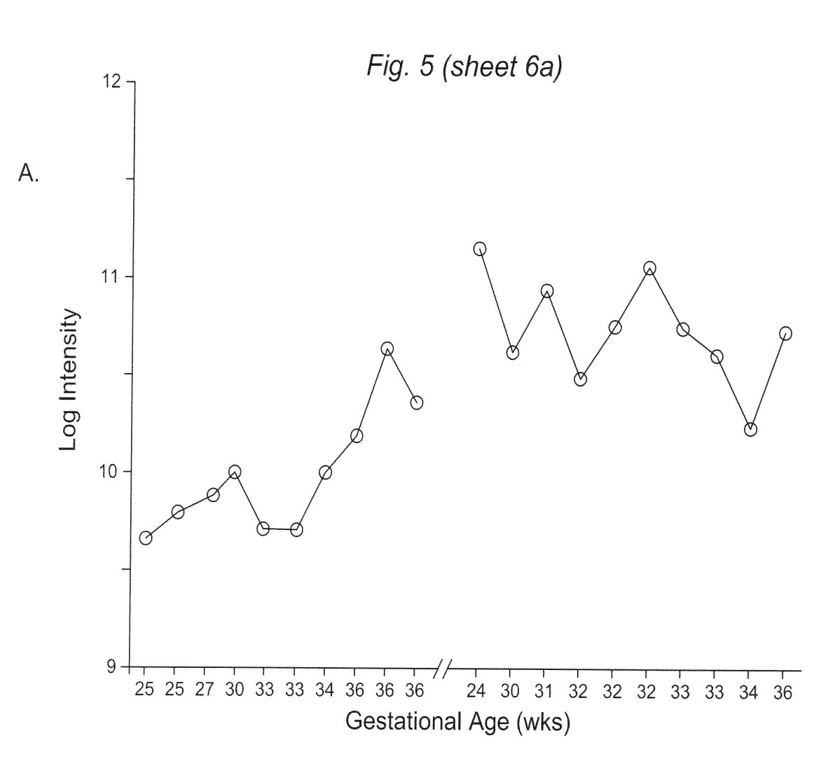

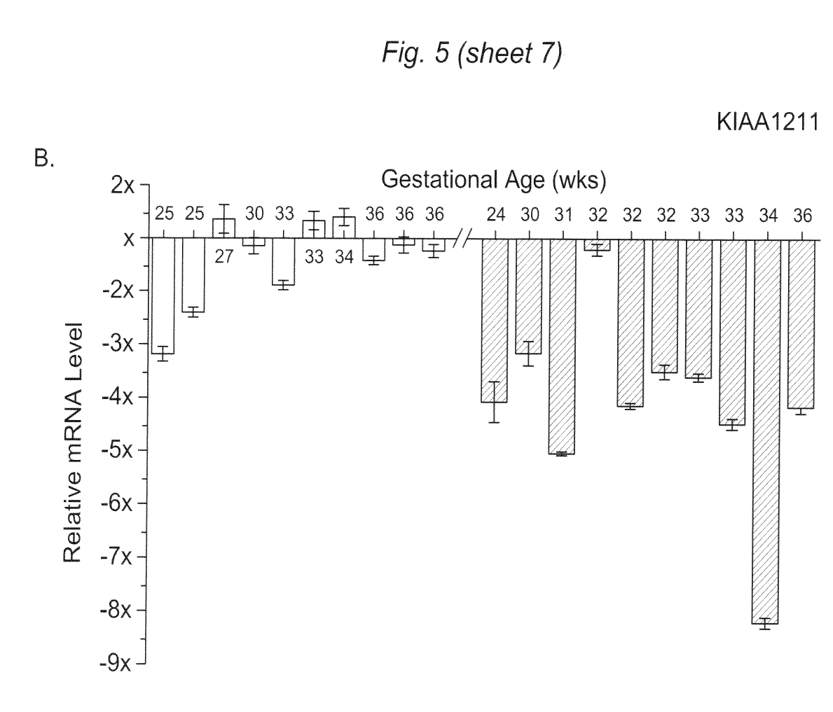

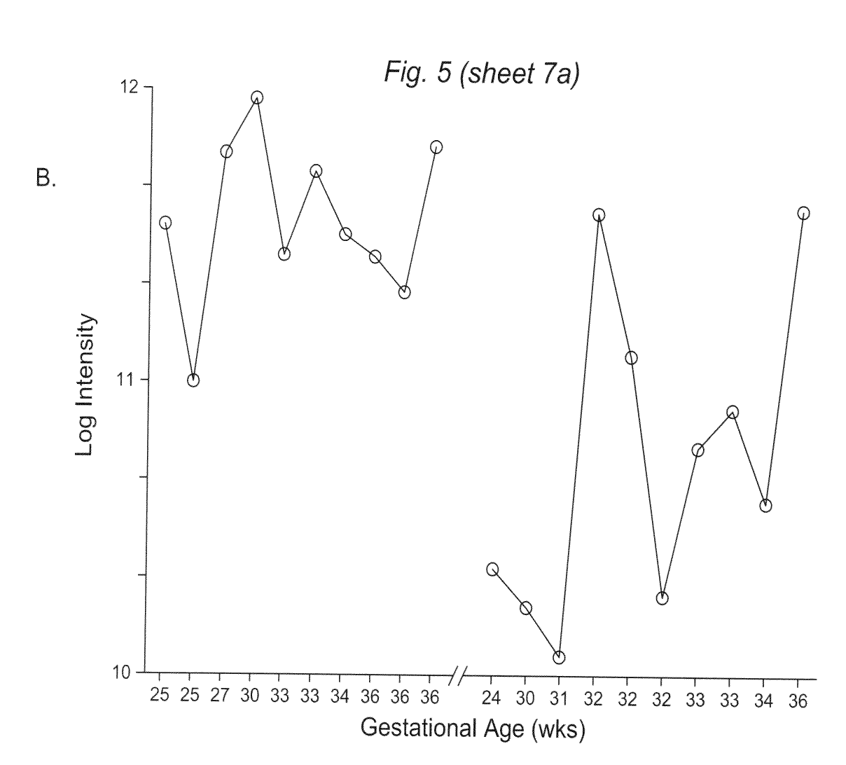

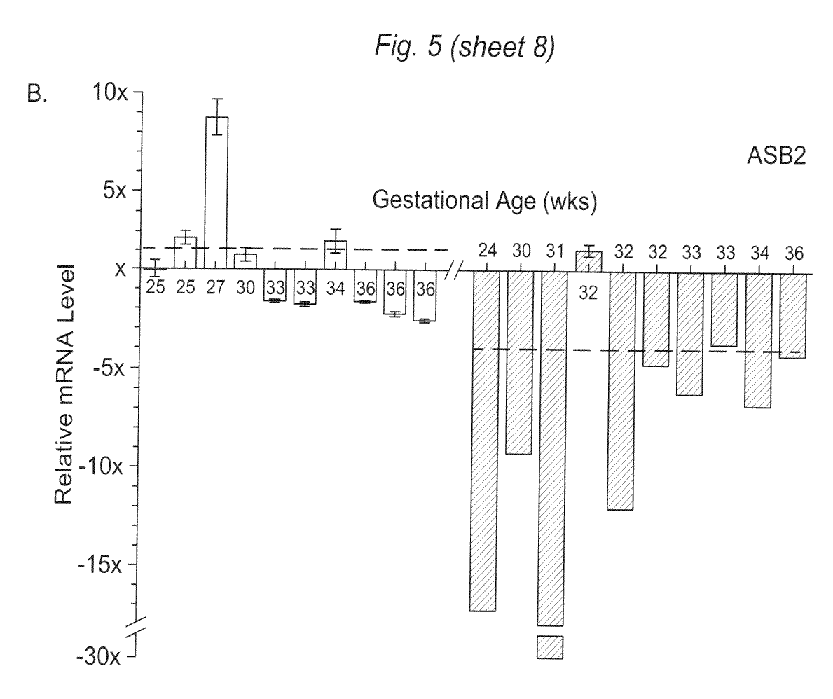

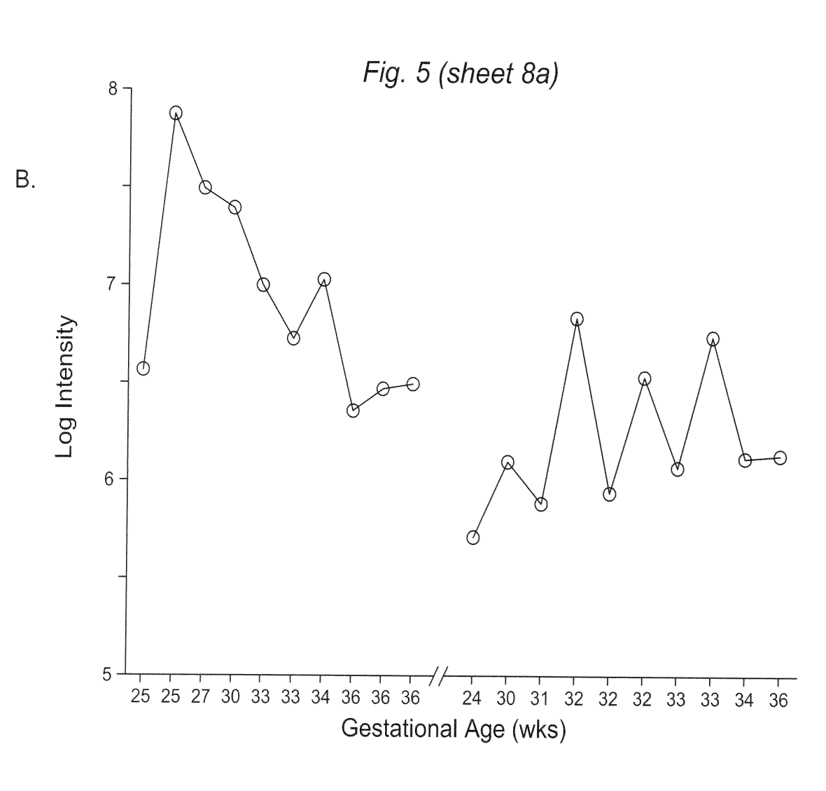

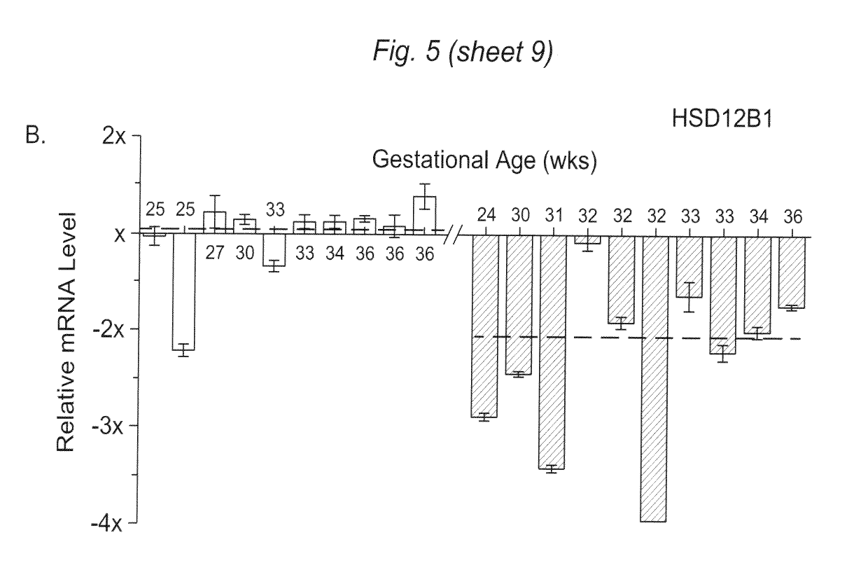

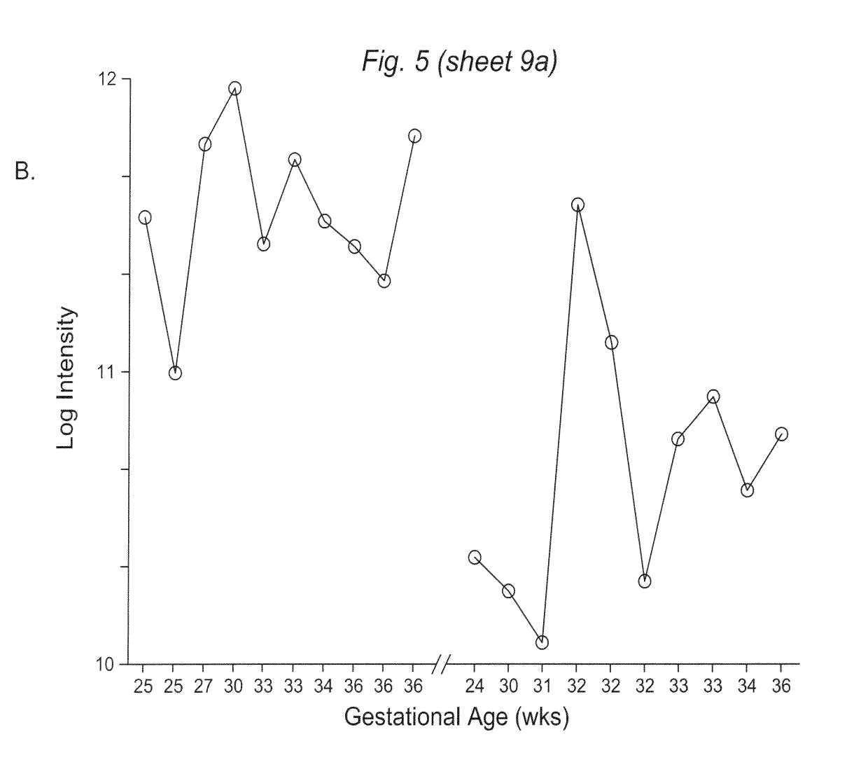

To further validate the microarray data, we utilized two approaches: quantitative PCR (Q-PCR) to assess relative mRNA levels and immunolocalization to confirm differential expression at the protein level. FIG. 5 shows the changes in expression of nine genes identified as being either upregulated or downregulated in normal and preeclampsia basal plate as a function of gestational age.

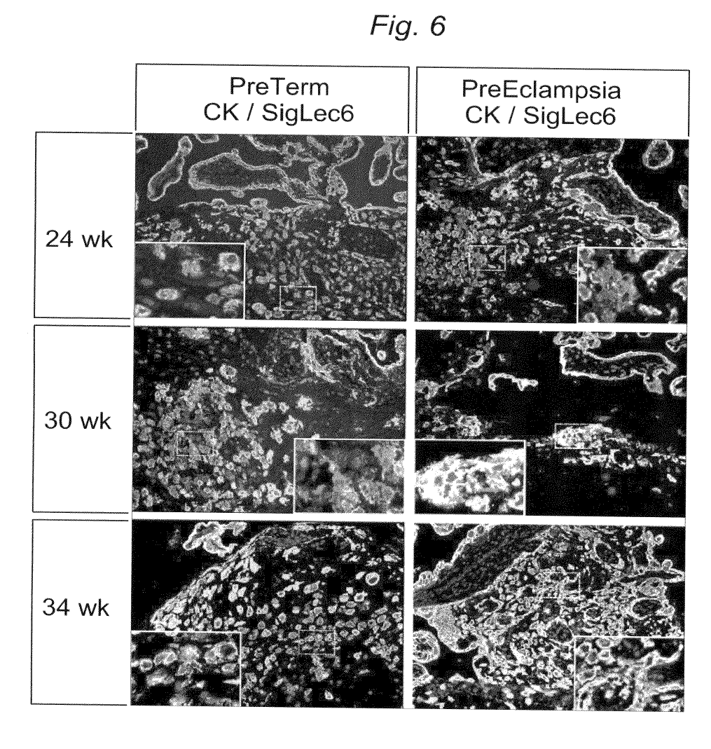

A protein, termed SigLec-6 (OB-BP-1), not previously known to be associated with preeclampsia, was elevated 2.74 fold. This protein is a transmembrane leptin binding protein with no similarity to the Ob-R leptin receptor. SigLec-6 shows a restricted tissue expression pattern, with expression highest in placenta, and moderate expression in spleen, PBL, and small intestine. FIG. 6 shows the increased expression of SigLec-6 in preeclampsia placentas at 24, 30, and 34 weeks of gestation. These results point to an altered leptin pathway in preeclampsia and point to a molecular target for therapeutic intervention.

### Example 3

### Prediction of Risk for Developing Preeclampsia

The identification of the molecular markers of the present invention allow a physician to determine a patient's risk for the development of preeclampsia. Because multiple and distinct genes are upregulated or down regulated in preeclampsia, the particular expression pattern of one or more, or all, of these genes can serve as a molecular signature with which to predict the risk of development of preeclampsia in an otherwise asymptomatic patient. Accordingly, a placental biopsy or a sample of bodily fluid (e.g., blood, saliva, cervicovaginal fluid, or urine) is taken from a patient. The gene expression pattern of the sample for the markers of this invention (mRNA or the corresponding polypeptides) is determined. Based on the particular markers expressed, as well as their levels, a patient's risk for the development of preeclampsia is assessed.

### Example 4

### Preventative Strategies and Therapeutic Interventions Based on Preeclampsia Subtype

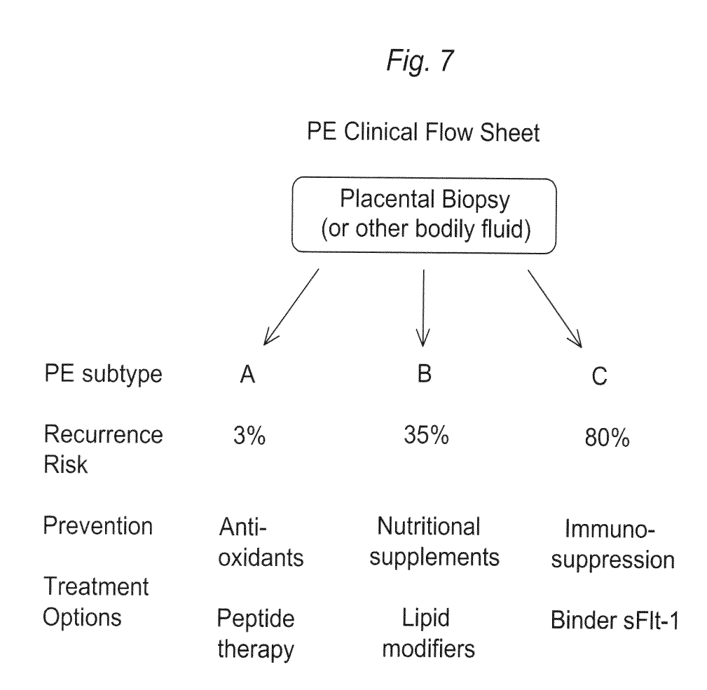

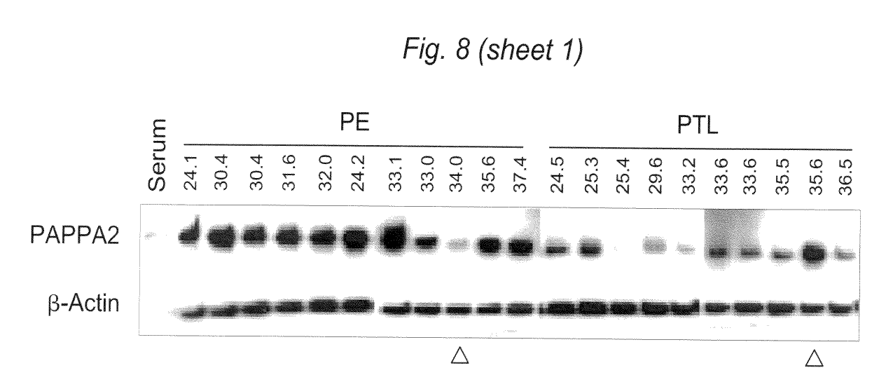

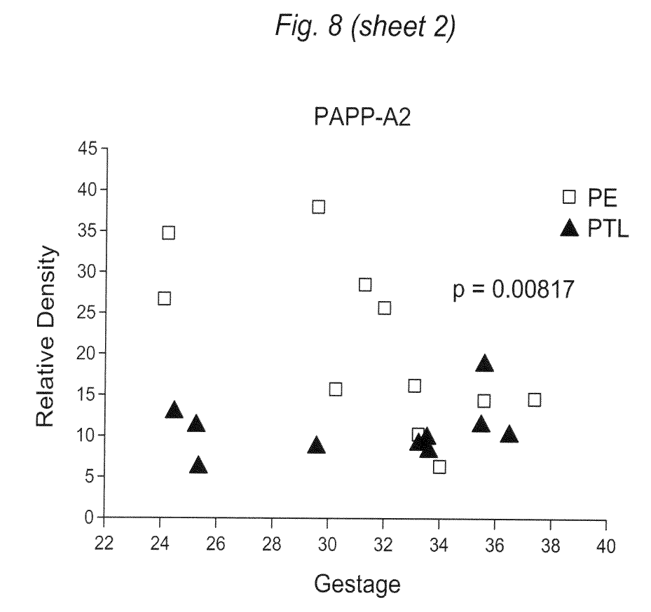

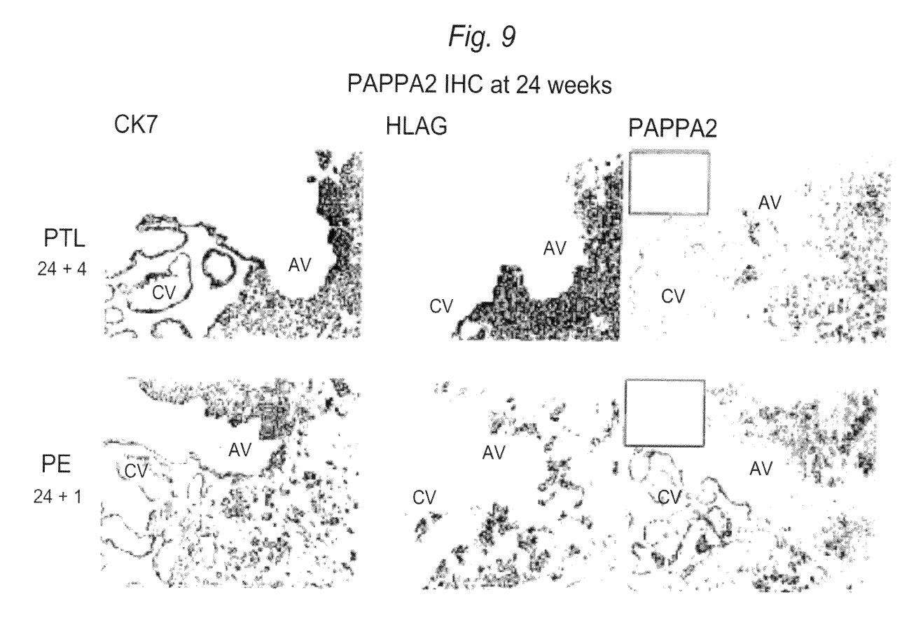

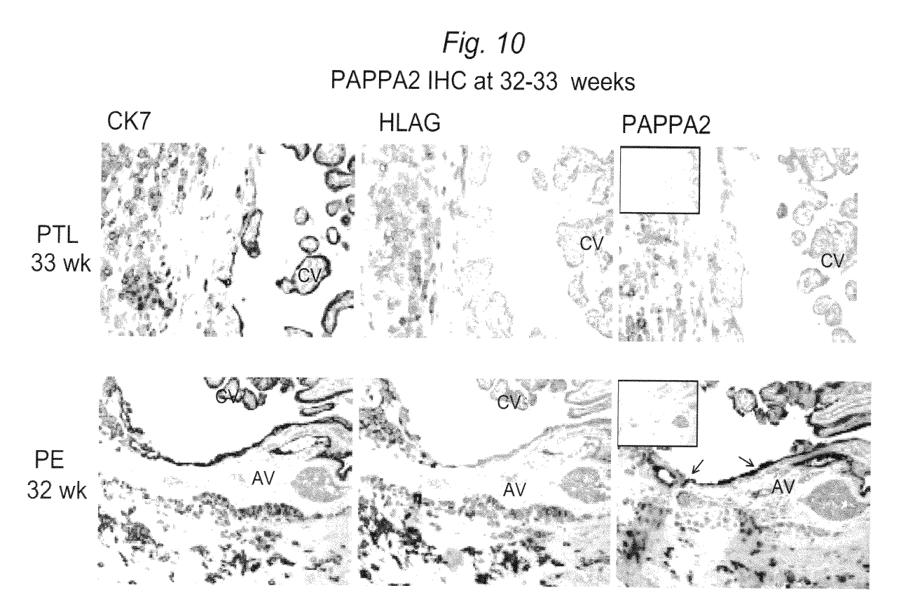

The identification of the molecular markers of the present invention in combination with close correlation with particular clinical manifestations of preeclampsia allow the subtyping of this disease and the fine tailoring of preventative strategies and therapeutic interventions based on subtype. Because multiple and distinct genes are upregulated or down regulated in preeclampsia, the particular expression pattern of one or more, or all, of these genes can serve as a molecular signature that defines a particular subtype of preeclampsia, with its own set of preferred preventative or therapeutic measures. Accordingly, a placental biopsy or a sample of bodily fluid (e.g., blood, saliva, cervicovaginal fluid, or urine) is taken from a patient. The gene expression pattern of the sample for the markers of this invention (mRNA or the corresponding polypeptides) is determined. Based on the particular markers expressed, as well as their levels, a patient with preeclampsia is assigned to a particular subtype for which a rate of recurrence is known and a preferred method of prevention or treatment is known. An example of such a preeclampsia clinical flow sheet is shown in FIG. 7.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of diagnosing preeclampsia in a subject, the method comprising the steps of:
(a) contacting a biological sample from the subject with a reagent that specifically binds to at least one marker selected from the group consisting of the nucleic acid and corresponding protein sequences shown in FIG. 3, FIG. 4, or Table 4; and
(b) determining whether or not the marker is overexpressed or underexpressed in the sample; thereby providing a diagnosis for preeclampsia.

2. The method of claim 1, wherein the reagent is an antibody.

3. The method of claim 2, wherein the antibody is monoclonal.

4. The method of claim 1, wherein the reagent is a nucleic acid.

5. The method of claim 1, wherein the reagent is an oligonucleotide.

6. The method of claim 1, wherein the reagent is an RT PCR primer set.

7. The method of claim 1, wherein the sample is a placental biopsy.

8. The method of claim 1, wherein the sample is a blood sample.

9. The method of claim 1, wherein the sample is a urine sample.

10. The method of claim 1, wherein the sample is a saliva sample.

11. The method of claim 1, wherein the sample is cervicovaginal fluid.

12. A method of providing a prediction for the risk for developing preeclampsia in a subject, the method comprising the steps of:
a) contacting a biological sample from the subject with a reagent that specifically binds to at least one marker selected from the group consisting of the nucleic acid and corresponding protein sequences shown in FIG. 3, FIG. 4, or Table 4; and
(b) determining whether or not the marker is overexpressed or underexpressed in the sample; thereby providing a prediction for preeclampsia development.

13. The method of claim 12, wherein the reagent is an antibody.

14. The method of claim 13, wherein the antibody is monoclonal.

15. The method of claim 12, wherein the reagent is a nucleic acid.

16. The method of claim 12, wherein the reagent is an oligonucleotide.

17. The method of claim 12, wherein the reagent is an RT PCR primer set.

18. The method of claim 12, wherein the sample is a placental biopsy.

19. The method of claim 12, wherein the sample is a blood sample.

20. The method of claim 12, wherein the sample is a urine sample.

21. The method of claim 12, wherein the sample is a saliva sample.

22. The method of claim 12, wherein the sample is cervicovaginal fluid.

23. The method of claim 12, wherein the prediction is a prognosis for the development of preeclampsia during a preexisting pregnancy or recurrence of preeclampsia during a subsequent pregnancy.

24. A method of identifying a compound that prevents or treats preeclampsia, the method comprising the steps of:
(a) contacting a compound with a sample comprising a cell that expresses a marker selected from the group consisting of the nucleic acid and corresponding protein sequences shown in FIG. 3, FIG. 4, or Table 4; and
(b) determining the functional effect of the compound on the marker, thereby identifying a compound that prevents or treats preeclampsia.

25. The method of claim 24, wherein the compound is a small molecule.

26. The method of claim 24, wherein the compound is a siRNA.

27. The method of claim 24, wherein the compound is a ribozyme.

28. The method of claim 24, wherein the compound is an antibody.

29. The method of claim 28, wherein the antibody is monoclonal.
